SEQUENCE LISTING (48 sequences)

SEQ ID NO: 1 - protein, 183 aa, Homo sapiens
MERVQPLEENVGNAARPRFERNKLLLVASVIQGLGLLLCFTYICLHFSALMVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - DNA, 552 bp, Homo sapiens
atggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttatggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 3 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 4 - DNA, 597 bp, Mus musculus
atggaaggggaaggggttcaacccctggatgagaatctggaaaacggatcaaggccaagattcaagtggaagaagacgctaaggctggtggtctctgggatcaagggagcagggatgcttctgtgcttcatctatgtctgcctgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtga

SEQ ID NO: 5 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 6 - DNA, 3657 bp, Homo sapiens
atgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatga

SEQ ID NO: 7 - protein, 1218 aa, Mus musculus
MRSPRTRGRPGRPLSLLLALLCALRAKVCGASGQFELEILSMQNVNGELQNGNCCGGVRNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPDCNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTTCEVIDSCTVAMASNDTPEGVRYISSNVCGPHGKCKSQSGGKFTCDCNKGFTGTYCHENINDCESNPCKNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCRLHKSHNECPSGQSCIPVLDDQCFVRPCTGVGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDFLVPLLSSVLTVAWVCCLVTAFYWCVRKRRKPSSHTHSAPEDNTTNNVREQLNQIKNPIEKHGANTVPIKDYENKNSKMSKIRTHNSEVEEDDMDKHQQKVRFAKQPVYTLVDREEKAPSGTPTKHPNWTNKQDNRDLESAQSLNRMEYIV

SEQ ID NO: 8 - DNA, 3657 bp, Mus musculus
atgcggtccccacggacgcgcggccggcccgggcgccccctgagtcttctgctcgccctgctctgtgccctgcgagccaaggtgtgcggggcctcgggtcagtttgagctggagatcctgtccatgcagaacgtgaatggagagctacagaatgggaactgttgtggtggagtccggaaccctggcgaccgcaagtgcacccgcgacgagtgtgatacgtacttcaaagtgtgcctcaaggagtatcagtcccgcgtcactgccgggggaccctgcagcttcggctcagggtctacgcctgtcatcgggggtaacaccttcaatctcaaggccagccgtggcaacgaccgtaatcgcatcgtactgcctttcagtttcgcctggccgaggtcctacactttgctggtggaggcctgggattccagtaatgacactattcaacctgatagcataattgaaaaggcttctcactcaggcatgataaaccctagccggcaatggcagacactgaaacaaaacacagggattgcccacttcgagtatcagatccgagtgacctgtgatgaccactactatggctttggctgcaataagttctgtcgtcccagagatgacttctttggacattatgcctgtgaccagaacggcaacaaaacttgcatggaaggctggatgggtcctgattgcaacaaagctatctgccgacagggctgcagtcccaagcatgggtcttgtaaacttccaggtgactgcaggtgccagtacggttggcagggcctgtactgcgacaagtgcatcccgcacccaggatgtgtccacggcacctgcaatgaaccctggcagtgcctctgtgagaccaactggggtggacagctctgtgacaaagatctgaattactgtgggactcatcagccctgtctcaaccggggaacatgtagcaacactgggcctgacaaataccagtgctcctgcccagagggctactcgggccccaactgtgaaattgctgagcatgcttgtctctctgacccctgccataaccgaggcagctgcaaggagacctcctcaggctttgagtgtgagtgttctccaggctggactggccccacgtgttccacaaacatcgatgactgttctccaaataactgttcccatgggggcacctgccaggatctggtgaatggattcaagtgtgtgtgcccgccccagtggactggcaagacttgtcagttagatgcaaatgagtgcgaggccaaaccttgtgtaaatgccagatcctgtaagaatctgattgccagctactactgtgattgccttcctggctggatgggtcagaactgtgacataaatatcaatgactgccttggccagtgtcagaatgacgcctcctgtcgggatttggttaatggttatcgctgtatctgtccacctggctatgcaggcgatcactgtgagagagacatcgatgagtgtgctagcaacccctgcttgaatgggggtcactgtcagaatgaaatcaacagattccagtgtctctgtcccactggtttctctggaaacctctgtcagctggacatcgattactgcgagcccaacccttgccagaatggcgcccagtgctacaatcgtgccagtgactatttctgcaagtgccccgaggactatgagggcaagaactgctcacacctgaaagaccactgccgtaccaccacctgcgaagtgattgacagctgcactgtggccatggcctccaacgacacgcctgaaggggtgcggtatatctcttctaacgtctgtggtccccatgggaagtgcaagagccagtcgggaggcaaattcacctgtgactgtaacaaaggcttcaccggcacctactgccatgaaaatatcaacgactgcgagagcaacccctgtaaaaacggtggcacctgcatcgatggcgttaactcctacaagtgtatctgtagtgacggctgggagggagcgcactgtgagaacaacataaatgactgtagccagaacccttgtcactacgggggtacatgtcgagacctggtcaatgacttttactgtgactgcaaaaatggctggaaaggaaagacttgccattcccgtgacagccagtgtgacgaagccacgtgtaataatggtggtacctgctatgatgaagtggacacgtttaagtgcatgtgtcccggtggctgggaaggaacaacctgtaatatagctagaaacagtagctgcctgccgaacccctgtcataatggaggtacctgcgtggtcaatggagactccttcacctgtgtctgcaaagaaggctgggaggggcctatttgtactcaaaataccaacgactgcagtccccatccttgttacaatagcgggacctgtgtggacggagacaactggtatcggtgcgaatgtgccccgggttttgctgggccagactgcaggataaacatcaatgagtgccagtcttccccttgtgcctttggggccacctgtgtggatgagatcaatggctaccagtgtatctgccctccaggacatagtggtgccaagtgccatgaagtttcagggcgatcttgcatcaccatggggagagtgatacttgatggggccaagtgggatgatgactgtaacacctgccagtgcctgaatggacgggtggcctgctccaaggtctggtgtggcccgagaccttgcaggctccacaaaagccacaatgagtgccccagtgggcagagctgcatcccggtcctggatgaccagtgtttcgtgcgcccctgcactggtgttggcgagtgtcggtcctccagcctccagccagtgaagaccaagtgcacatctgactcctattaccaggataactgtgcaaacatcactttcacctttaacaaagagatgatgtctccaggtcttaccaccgaacacatttgcagcgaattgaggaatttgaatatcctgaagaatgtttctgctgaatattcgatctacatagcctgtgagccttccctgtcagcaaacaatgaaatacacgtggccatctctgcagaagacatccgggatgatgggaaccctgtcaaggaaattaccgataaaataatagatctcgttagtaaacgggatggaaacagctcacttattgctgcggttgcagaagtcagagttcagaggcgtcctctgaaaaacagaacagatttcctggttcctctgctgagctctgtcttaacagtggcttgggtctgttgcttggtgacagccttctactggtgtgtaaggaagcggcggaagcccagcagccacactcactccgcccccgaggacaacaccaccaacaatgtgcgggagcagctgaaccaaatcaaaaaccccatcgagaaacacggagccaacacggtccccattaaggattacgagaacaaaaactcgaaaatgtcaaaaatcaggacacacaactcggaagtggaggaggatgacatggataaacaccagcagaaagtccgctttgccaaacagccagtgtatacgctggtagacagagaggagaaggcccccagcggcacgccgacaaaacacccgaactggacaaataaacaggacaacagagacttggaaagtgcccagagcttgaaccggatggaatacatcgtatag

SEQ ID NO: 9 - protein, 1404 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLDKTHTCPPCPQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 4275 bp, Homo sapiens
atgtacaggatgcaactcctgtcttgcattgcactaagtcttgcacttgtcacgaattcgatggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttgggcacggtgggcatgtgtgagttttgtccagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 11 - protein, 1424 aa, Mus musculus
QLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPLGCKPCICTQFELEILSMQNVNGELQNGNCCGGVRNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPDCNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTTCEVIDSCTVAMASNDTPEGVRYISSNVCGPHGKCKSQSGGKFTCDCNKGFTGTYCHENINDCESNPCKNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCRLHKSHNECPSGQSCIPVLDDQCFVRPCTGVGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDGNSISAMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - DNA, 4335 bp, Mus musculus
atgtacaggatgcaactcctgtcttgcattgcactaagtcttgcacttgtcacgaattcgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgggttgtaagccttgcatatgtacacagtttgagctggagatcctgtccatgcagaacgtgaatggagagctacagaatgggaactgttgtggtggagtccggaaccctggcgaccgcaagtgcacccgcgacgagtgtgatacgtacttcaaagtgtgcctcaaggagtatcagtcccgcgtcactgccgggggaccctgcagcttcggctcagggtctacgcctgtcatcgggggtaacaccttcaatctcaaggccagccgtggcaacgaccgtaatcgcatcgtactgcctttcagtttcgcctggccgaggtcctacactttgctggtggaggcctgggattccagtaatgacactattcaacctgatagcataattgaaaaggcttctcactcaggcatgataaaccctagccggcaatggcagacactgaaacaaaacacagggattgcccacttcgagtatcagatccgagtgacctgtgatgaccactactatggctttggctgcaataagttctgtcgtcccagagatgacttctttggacattatgcctgtgaccagaacggcaacaaaacttgcatggaaggctggatgggtcctgattgcaacaaagctatctgccgacagggctgcagtcccaagcatgggtcttgtaaacttccaggtgactgcaggtgccagtacggttggcagggcctgtactgcgacaagtgcatcccgcacccaggatgtgtccacggcacctgcaatgaaccctggcagtgcctctgtgagaccaactggggtggacagctctgtgacaaagatctgaattactgtgggactcatcagccctgtctcaaccggggaacatgtagcaacactgggcctgacaaataccagtgctcctgcccagagggctactcgggccccaactgtgaaattgctgagcatgcttgtctctctgacccctgccataaccgaggcagctgcaaggagacctcctcaggctttgagtgtgagtgttctccaggctggactggccccacgtgttccacaaacatcgatgactgttctccaaataactgttcccatgggggcacctgccaggatctggtgaatggattcaagtgtgtgtgcccgccccagtggactggcaagacttgtcagttagatgcaaatgagtgcgaggccaaaccttgtgtaaatgccagatcctgtaagaatctgattgccagctactactgtgattgccttcctggctggatgggtcagaactgtgacataaatatcaatgactgccttggccagtgtcagaatgacgcctcctgtcgggatttggttaatggttatcgctgtatctgtccacctggctatgcaggcgatcactgtgagagagacatcgatgagtgtgctagcaacccctgcttgaatgggggtcactgtcagaatgaaatcaacagattccagtgtctctgtcccactggtttctctggaaacctctgtcagctggacatcgattactgcgagcccaacccttgccagaatggcgcccagtgctacaatcgtgccagtgactatttctgcaagtgccccgaggactatgagggcaagaactgctcacacctgaaagaccactgccgtaccaccacctgcgaagtgattgacagctgcactgtggccatggcctccaacgacacgcctgaaggggtgcggtatatctcttctaacgtctgtggtccccatgggaagtgcaagagccagtcgggaggcaaattcacctgtgactgtaacaaaggcttcaccggcacctactgccatgaaaatatcaacgactgcgagagcaacccctgtaaaaacggtggcacctgcatcgatggcgttaactcctacaagtgtatctgtagtgacggctgggagggagcgcactgtgagaacaacataaatgactgtagccagaacccttgtcactacgggggtacatgtcgagacctggtcaatgacttttactgtgactgcaaaaatggctggaaaggaaagacttgccattcccgtgacagccagtgtgacgaagccacgtgtaataatggtggtacctgctatgatgaagtggacacgtttaagtgcatgtgtcccggtggctgggaaggaacaacctgtaatatagctagaaacagtagctgcctgccgaacccctgtcataatggaggtacctgcgtggtcaatggagactccttcacctgtgtctgcaaagaaggctgggaggggcctatttgtactcaaaataccaacgactgcagtccccatccttgttacaatagcgggacctgtgtggacggagacaactggtatcggtgcgaatgtgccccgggttttgctgggccagactgcaggataaacatcaatgagtgccagtcttccccttgtgcctttggggccacctgtgtggatgagatcaatggctaccagtgtatctgccctccaggacatagtggtgccaagtgccatgaagtttcagggcgatcttgcatcaccatggggagagtgatacttgatggggccaagtgggatgatgactgtaacacctgccagtgcctgaatggacgggtggcctgctccaaggtctggtgtggcccgagaccttgcaggctccacaaaagccacaatgagtgccccagtgggcagagctgcatcccggtcctggatgaccagtgtttcgtgcgcccctgcactggtgttggcgagtgtcggtcctccagcctccagccagtgaagaccaagtgcacatctgactcctattaccaggataactgtgcaaacatcactttcacctttaacaaagagatgatgtctccaggtcttaccaccgaacacatttgcagcgaattgaggaatttgaatatcctgaagaatgtttctgctgaatattcgatctacatagcctgtgagccttccctgtcagcaaacaatgaaatacacgtggccatctctgcagaagacatccgggatgatgggaaccctgtcaaggaaattaccgataaaataatagatctcgttagtaaacgggatggaaacagctcacttattgctgcggttgcagaagtcagagttcagaggcgtcctctgaaaaacagaacagatgggaattcgatatcggccatggttagatctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 13 - protein, 10 aa, Artificial Sequence
  example of polypetide linker
DKTHTCPPCP

SEQ ID NO: 14 - DNA, 32 bp, Artificial Sequence
  sense primer Fc-OX40L-ecto-F
gcgcgaattcgcaactctcttcctctccggca

SEQ ID NO: 15 - DNA, 45 bp, Artificial Sequence
  anti-sense primer pFUSE-OX40L-Linker-R
tgtacatatgcaaggcttacaacccagtggtacttggttcacagt

SEQ ID NO: 16 - DNA, 48 bp, Artificial Sequence
  sense primer Fc-linker-JAG1-ecto-F
ggttgtaagccttgcatatgtacacagtttgagctggagatcctgtcc

SEQ ID NO: 17 - DNA, 36 bp, Artificial Sequence
  anti-sense primer Fc-JAG1-ecto R
gcgcgaattcccatctgttctgtttttcagaggacg

SEQ ID NO: 18 - DNA, 32 bp, Artificial Sequence; identical to SEQ ID NO: 14
  sense primer Fc-OX40L-ecto-F

SEQ ID NO: 19 - DNA, 36 bp, Artificial Sequence; identical to SEQ ID NO: 17
  anti-sense primer Fc-JAG1-ecto R

SEQ ID NO: 20 - DNA, 31 bp, Artificial Sequence
  primer pet15b-OX40L-F
gcgccatatgcaactctcttcctctccggca

SEQ ID NO: 21 - DNA, 28 bp, Artificial Sequence
  reverse primer pet15b-Fc-R
gcgcggatcctcatttaccaggagagtg

SEQ ID NO: 22 - DNA, 37 bp, Artificial Sequence
  reverse primer pet15b-JAG1-R
gcgcggatcctcaatctgttctgtttttcagaggacg

SEQ ID NO: 23 - DNA, 37 bp, Artificial Sequence
  primer Bacu-OX40L-F
cgcgggatccaccatgcaactctcttcctctccggca

SEQ ID NO: 24 - DNA, 33 bp, Artificial Sequence
  Bacu-Reverse primer
cgcggcggccgcccagctagcgacactgggatc

SEQ ID NO: 25 - protein, 9 aa, Artificial Sequence
  mouse IgG1 signature peptide
DVLTITLTP

SEQ ID NO: 26 - protein, 17 aa, Artificial Sequence
  mouse IgG1 signature peptide
NTQPIMDTDGSYFVYSK

SEQ ID NO: 27 - DNA, 32 bp, Artificial Sequence
  the sense OX40L primer
taaggaatccgctccactgtgtcggggacacc

SEQ ID NO: 28 - DNA, 53 bp, Artificial Sequence
  anti-sense OX40L primer
tgggcacggtgggcatgtgtgagttttgtccgcacggcccccggggacctcca

SEQ ID NO: 29 - DNA, 53 bp, Artificial Sequence
  sense Jagged-1 primer
cagttcgagttggagatcctgtcgacaaaactcacacatgcccaccgtgccca

SEQ ID NO: 30 - DNA, 35 bp, Artificial Sequence
  anti-sense Jagged-1 primer
tgctgatatcccatctgttctgttcttcagaggcc

SEQ ID NO: 31 - DNA, 36 bp, Artificial Sequence
  primer pet15b-OX40L-F
acttcatatgatggtatcacatcggtatcctcgaat

SEQ ID NO: 32 - DNA, 38 bp, Artificial Sequence
  reverse primer pet15b-Fc-R
ctagggatccttatcatttacccggagacagggagagg

SEQ ID NO: 33 - DNA, 37 bp, Artificial Sequence
  reverse primer pet15b-JAG1-R
ctagggatccttaatctgttctgttcttcagaggccg

SEQ ID NO: 34 - DNA, 42 bp, Artificial Sequence
  forward primer pFUSE-chimera
acttctcgagaccatgtacaggatgcaactcctgtcttgcat

SEQ ID NO: 35 - DNA, 39 bp, Artificial Sequence
  forward primer pFUSE-chimera
ctagaaagctttcatttacccggagacagggagaggctc

SEQ ID NO: 36 - protein, 25 aa, Artificial Sequence
  human JAG1 signature peptide
VTAGGPCSFGSGSTPVIGGNTFNLK

SEQ ID NO: 37 - protein, 12 aa, Artificial Sequence
  human JAG1 signature peptide
NTGVAHFEYQIR

SEQ ID NO: 38 - protein, 11 aa, Artificial Sequence
  human JAG1 signature peptide
DLVNDFYCDCK

SEQ ID NO: 39 - protein, 17 aa, Artificial Sequence
  human JAG1 signature peptide
EMMSPGLTTEHICSELR

SEQ ID NO: 40 - protein, 22 aa, Artificial Sequence
  human IgG1-Fc signature peptide
TPEVTCVVVDVSHEDPEVKFNW

SEQ ID NO: 41 - protein, 11 aa, Artificial Sequence
  uman IgG1-Fc signature peptide
YVDGVEVHNAK

SEQ ID NO: 42 - protein, 8 aa, Artificial Sequence
  example of polypetide linker
GCKPCICT

SEQ ID NO: 43 - DNA, 32 bp, Artificial Sequence
  sense primer for truncated chimeric OX40L-Jagged-1-Fc
gcgcgatatcgcaactctcttcctctccggca

SEQ ID NO: 44 - DNA, 30 bp, Artificial Sequence
  anti-sense primer for truncated chimeric OX40L-Jagged-1-Fc
gcgcccatggcttcacagttggggcccgag

SEQ ID NO: 45 - DNA, 36 bp, Artificial Sequence
  sense primer for truncated chimeric OX40L-Jagged-1-Fc
gacgacgacaagatgcaactctcttcctctccggca

SEQ ID NO: 46 - DNA, 35 bp, Artificial Sequence
  anti-sense primer for truncated chimeric OX40L-Jagged-1-Fc
gaggagaagcccggttcacagttggggcccgagta

SEQ ID NO: 47 - DNA, 40 bp, Artificial Sequence
  sense primer for truncated chimeric OX40L-Jagged-1-Fc
ccttgatatcgatgtacaggatgcaactcctgtcttgcat

SEQ ID NO: 48 - DNA, 33 bp, Artificial Sequence
  anti-sense primer for truncated chimeric OX40L-Jagged-1-Fc
ggctccatggcttcacagttgggtcctgaatac

(19) United States
(12) Patent Application Publication
(10) Pub. No.: US 2018/0320135 A1
(43) Pub. Date: Nov. 8, 2018

(54) OX40L-JAGGED-1 CHIMERIC POLYPEPTIDES AND USES THEREOF

(71) Applicant: THE BOARD OF TRUSTEES OF THE UNIVERSITY OF ILLINOIS, Urbana, IL (US)

(72) Inventors: Bellur S. Prabhakar, Oak Brook, IL (US); Palash Bhattacharya, Chicago, IL (US); Prabhakaran Kumar, Chicago, IL (US)

(21) Appl. No.: 15/773,443

(22) PCT Filed: Nov. 3, 2016
(86) PCT No.: PCT/US2016/060349

§ 371 Date: May 3, 2018

Related U.S. Application Data
(60) Provisional application No. 62/252,203, filed on Nov. 6, 2015.

(51) Int. Cl. C12N 5/0783; C07K 14/705

(52) U.S. Cl. CPC C12N 5/0637; C07K 14/70575; C12N 2501/599; C07K 2319/30; C07K 14/70596

(57) ABSTRACT

This invention relates to chimeric polypeptides comprising OX40L and Jagged-1 polypeptides and fragments thereof and their uses for treatment of autoimmune diseases.

### STATEMENT OF GOVERNMENT INTEREST

This invention was made with Government support under grant number 5R01A1107516 awarded by the National Institutes of Health. The Government has certain rights in the invention.

### TECHNICAL FIELD

This application relates to the field of immunology. Particularly, this invention relates to chimeric polypeptides comprising OX40L and Jagged-1 polypeptides and their uses for treatment of autoimmune diseases.

### BACKGROUND OF THE INVENTION

Humans suffer from over one hundred different autoimmune diseases with very high associated morbidities. Patients with autoimmune diseases are subjected to life-long immunosuppressive or hormone replacement therapy. Although, immunomodulation using several biological agents such as anti-TNFα, anti-CD3, anti-B220, anti-CTLA4 have been developed they are non-specific, not curative and are accompanied by severe side effects. Therefore, harnessing the potential of regulatory T cells (Tregs) to promote peripheral tolerance is of immense clinical value. However, generating Tregs in vivo is very challenging because current Treg expansion methods involve T cell receptor (TCR) mediated activation which also causes effector T cell (Teff) proliferation. Therefore, the TCR based approaches can only be used for ex vivo expansion of Tregs which can then be infused into the patient, which is impractical for common clinical use. Thus, an effective method for treating autoimmune diseases using Treg cells is still needed.

### SUMMARY OF THE INVENTION

In some aspects, provided herein are chimeric polypeptides comprising a first and a second polypeptide, wherein one of the polypeptides is an OX40L polypeptide and one of the polypeptides is a Jagged-1 polypeptide. In particular embodiments, the chimeric peptide of the disclosure further comprises a linker. In other particular embodiments the first polypeptide is an OX40L polypeptide and the second polypeptide is a Jagged-1 polypeptide. In other particular embodiments the first polypeptide is a Jagged-1 polypeptide and the second polypeptide is an OX40L polypeptide.

In particular embodiments the OX40L polypeptide comprises the extracellular domain of OX40L or fragment thereof and the Jagged-1 polypeptide comprises the extracellular domain of Jagged-1 or fragment thereof.

In other particular embodiments the chimeric protein further comprises a Fc region of an immunoglobulin wherein the Fc domain comprises the CH2 and CH3 regions of the IgG heavy chain and the hinge region.

In other particular embodiments the linker comprises 10 amino acids from human immunoglobulin G1 hinge region. In other particular embodiments the linker comprises a polypeptide having SEQ ID NO: 13. In other particular embodiments the linker comprises a polypeptide having SEQ ID NO: 42.

In other aspects, provided herein are methods of treating an autoimmune disease in a patient in need of such treatment comprising administering to the patient a therapeutically effective amount of the chimeric polypeptides disclosed herein. In particular embodiments the autoimmune disease is an autoimmune thyroid disease such as Grave's disease or Hashimoto disease. In other embodiments the autoimmune disease is Type 1 Diabetes mellitus.

In other particular embodiments the patient is a human patient.

In some aspects, provided herein are methods of expanding T-regulatory cells comprising co-culturing said T-regulatory cells with the chimeric polypeptides disclosed herein.

### BRIEF DESCRIPTION OF THE DRAWINGS

FIG. 1. Scheme for construction of OX40L-JAGGED-1 chimeric cDNA construct by overlap extension PCR. OX40L (˜480 bp) and Jagged-1 (˜3.1 kb) cDNAs were individually PCR amplified with specific primers. The 3′ primer for OX40L and 5′ primer for Jagged-1 contained a common linker sequence of 24 nucleotides (˜8 amino acids). The cDNA PCR products were mixed in equimolar ratios. Overlap Extension PCR with 5′ OX40L forward primer and a 3′ Jagged-1 reverse primer was used to amplify OX40L-Jagged-1 chimeric cDNA (˜3.5 kb).

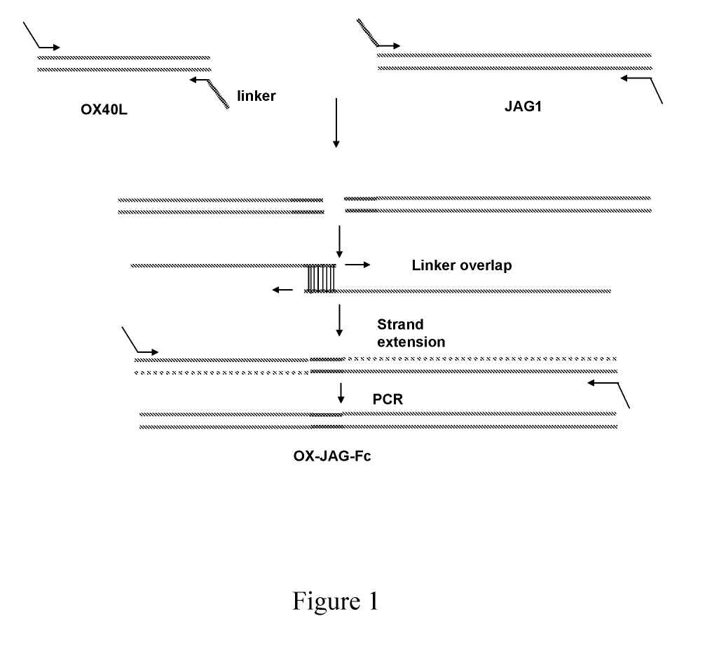

FIGS. 2A-2C. PCRs for cloning of OX40L-JAGGED-1 chimeric cDNA. FIG. 2A shows a PCR gel of an OX40L-specific PCR product with a 5′ Eco R1 restriction site (˜480 bp). FIG. 2B shows a PCR gel of a Jagged-1 specific PCR product with a 3′ Eco R1 restriction site (˜3.1 kB). FIG. 2C shows a PCR gel of an OX40L-Jagged-1 chimeric specific product.

FIG. 3. Verification of pFUSE cDNA clones by Restriction Digestion (EcoR1/Bg1 II). Restriction analysis of clones was done making use of two Bg1 II sites within the Jagged-1 sequence. While Eco R1 released the inserted chimeric fragment, Bg1 II further digested the insert in different sizes based on the construct cloned (˜500 bp for OX40L; ˜800 bp +˜2.2 kb for JAGGED-1; ˜1.3 kb+2.2 kb for chimeric OX40L-JAGGED-1). Digested vector is 4.2 kb in size.

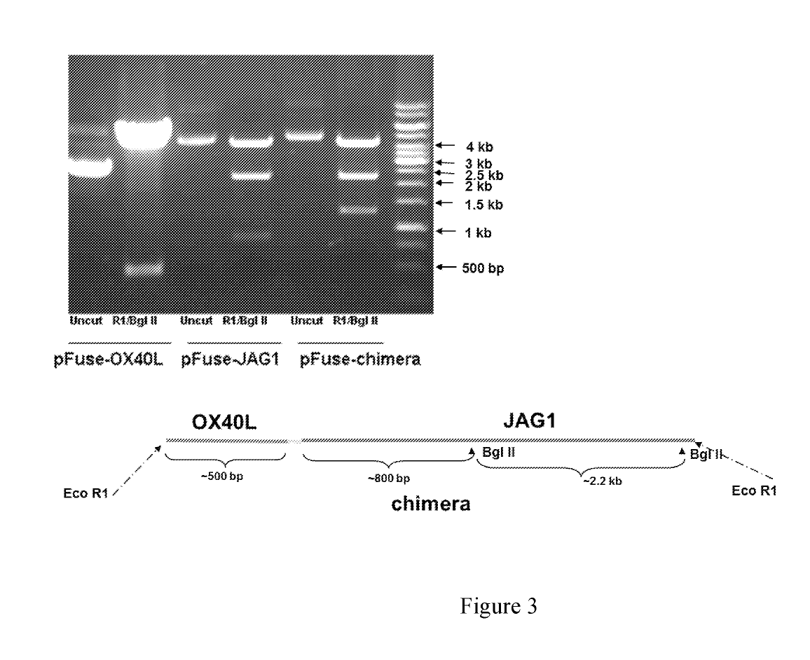

FIG. 4. Transfection and screening for expression of chimeric OX40L-JAGGED-1-Fc by Western blot. FIG. 4 shows a western blot gel for expression of chimeric OX40L-JAGGED-1-Fc in HEK 293 cells transfected with pFUSE- mOX40L-Fc, mJAGGED-1-Fc and mOX40L-JAGGED-1-Fc plasmids. Culture supernatants were collected and proteins were purified using protein A/G beads. Purified proteins were fractionated on 4-20% gradient SDS-PAGE, and Western blot analysis was performed using anti-mouse IgG antibodies. Expected molecular weight for OX40L-Fc-40 kDa; JAGGED-1-Fc˜140 kDa and OX40L-JAGGED-1-Fc˜160 kDa.

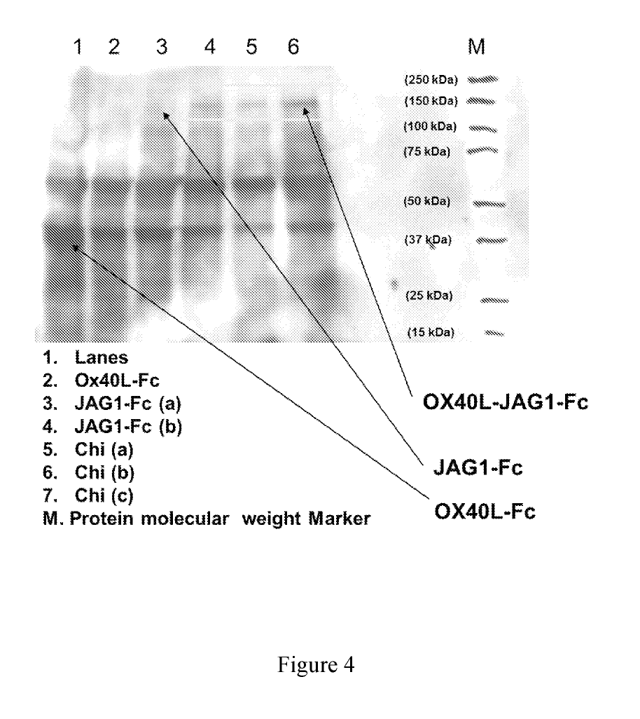

FIG. 5. Selecting stable clones by flow cytometry. Recombinant Chinese hamster ovary (CHO) cell clones (numbered E7, F8, E8 and F9) expressing mouse chimeric OX40L-Jagged-1 were analyzed by FACS. Cells were fixed, permeabilized and intracellularly stained with phycoerythrin (PE) coupled anti-mouse IgG antibodies.

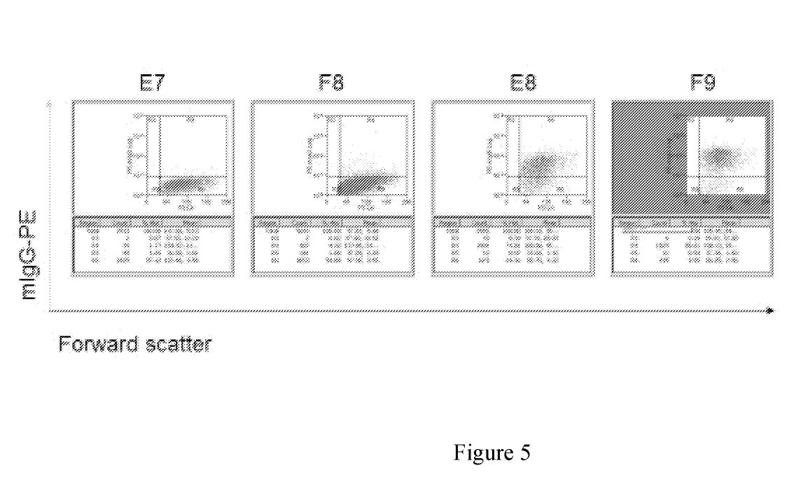

FIG. 6. Evaluating the capacity of OX40L-JAGGED-1-Fc to drive Treg proliferation ex vivo. CD4+T-cells were isolated from non-obese diabetic (NOD) mice and labeled with Celltrace. Celltrace labeled CD4+T-cells were cultured with splenic antigen presenting cells in the presence of different soluble OX40L-Fc or OX40L-JAGGED-1-Fc with or without IL-2. Celltrace dilution and Foxp3 expression was analyzed by FACS. This data suggests that recombinant Chimeric OX40L-JAGGED-1-Fc is capable of stimulating Foxp3 +Treg proliferation (top left quadrant in dot plots) in ex vivo cultures.

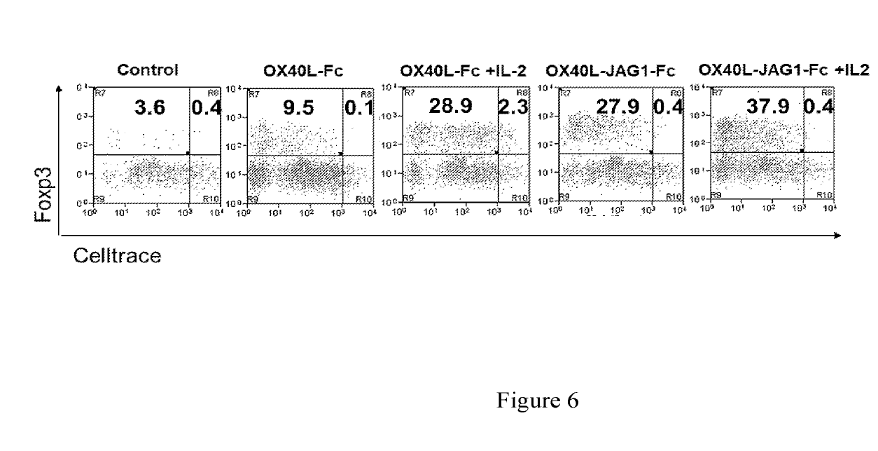

FIGS. 7A-7D. PCR amplification of OX40L and Jagged-1 ectodomains, and the chimeric product. FIG. 7A is a gel showing the PCR amplification of OX40L with a linker sequence. FIG. 7B is a gel showing the PCR amplification of Jagged-1 with linker sequence. FIG. 7C is a gel showing assembly linker PCR amplification of human OX40L-Jagged1-Fc chimera. FIG. 7D is a gel showing confirmation of pFUSE-chimera clone by restriction digestion with EcoRv.

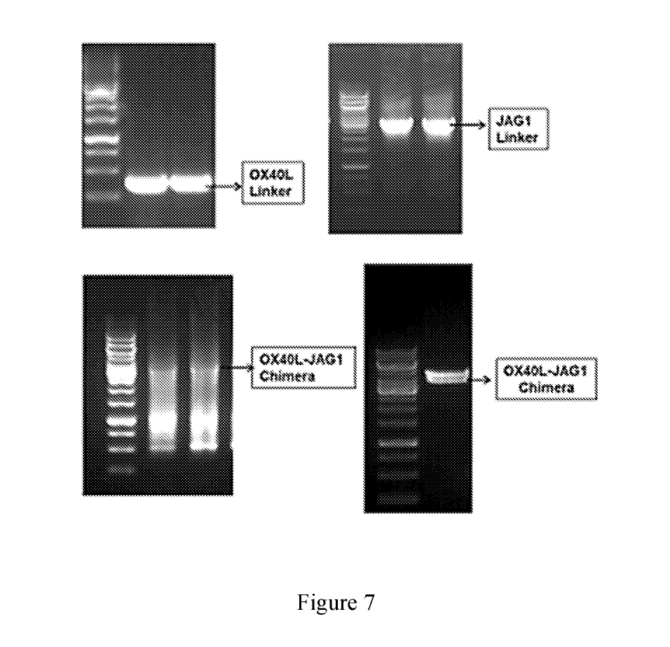

FIGS. 8A-8C. Expression of human chimeric protein. FIG. 8A is a FACS analysis of human OX40L-Jagged-1-Fc chimera producing HEK293T clone for chimera expression. FIG. 8B is a coomassie blue SDS-PAGE gel showing chimera protein band. FIG. 8C is a western blot analysis of chimera expression and secretion in lysate and cell culture supernatant from 293T cells expressing chimera protein.

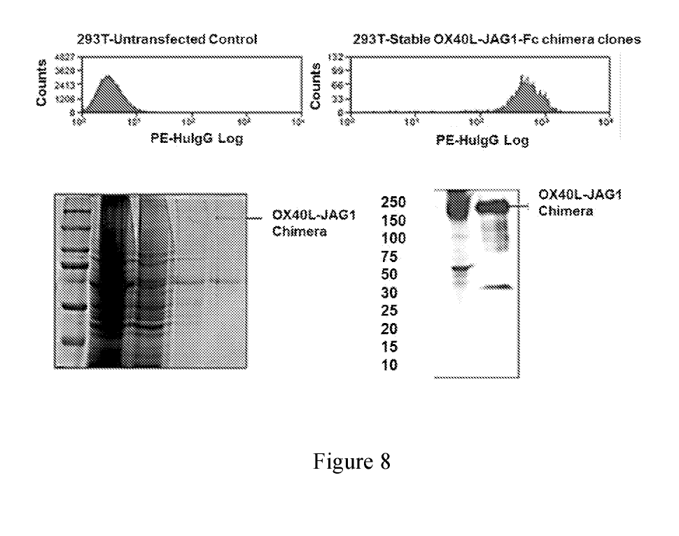

FIG. 9. Human Treg expansion induced by the human chimeric protein. Human CD4+ T-cells were labeled with Cell-Trace violet and treated with either IL-2 (IU/ml) or chimera (5μg/ml+IL-2) (25 IU/ml) for 5 days. After 5 days cells were stained with CD4-FITC, CD25-PE and FOXP3-FITC. CD4+ and CD4+CD25+ T cells were gated and proliferation was measured on the basis of CT-violet dilute. Percentages of resting and proliferating Treg cells are indicated in right and left upper quadrants respectively (n=3).

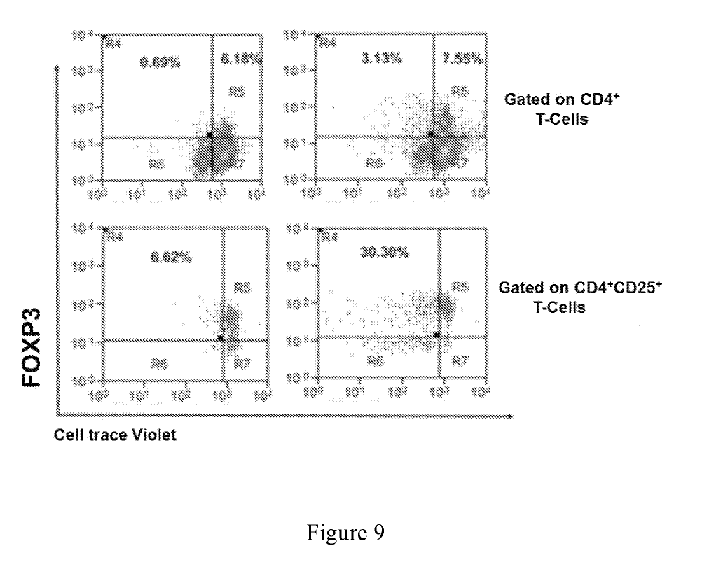

FIG. 10. Expression of truncated chimeric mOX40L-Jagged-1-Fc in 293 cells. FIG. 10 is a western blot gel illustrating expression of 1) OX40L-Fc, 2) full length chimeric mOX40L-Jagged-1-Fc, 3) truncated chimeric OX40L-Jagged-1-Fc (clone 1), 4) truncated chimeric OX40L-Jagged-1-Fc (clone 2) and 5) truncated chimeric OX40L-Jagged-1-Fc (clone 3). Briefly, HEK 293 cells were transfected with the plasmid pFUSE-trunc-chi (3 clones 1, 2 and 3). After 48-72 hours, supernatants were incubated with protein A/G agarose and bound proteins purified. Plasmids for expression of mOX40L alone or full length mOX40L-Jagged-1-Fc were also used as controls for protein expression. Purified proteins were resolved on SDS-PAGE and identified by western blot using anti-mouse IgG1 antibody. The expression level of the truncated construct was conspicuously greater than the full length construct.

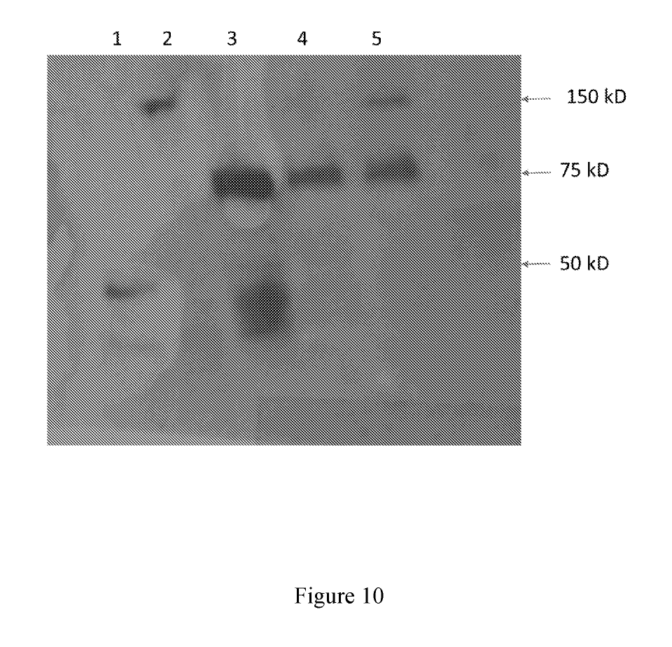

FIG. 11. Confirmation of pIEx-10 Ek/LIc-mOX40L-Jagged-1 clone by PCR. FIG. 11 is an agarose gel illustrating PCR amplification of cDNA of mouse truncated OX40L-Jagged-1-Fc chimera.

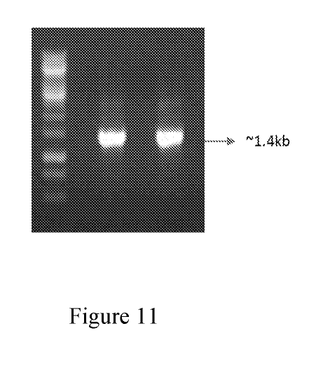

FIG. 12. Expression & purification of truncated mOX40L-Jagged-1 in sf8 insect cells. FIG. 12 is a Western Blot illustrating: Lane-1: Pop-culture product, 2: Flow through from Strep. Tactin column, 3: Wash from the column, 4: Final elute containing truncated mouse chimeric protein. Briefly, Sf9 cells were transfected with truncated chimera truncated pIEx-10 Ek/LIc-mOX40L-Jagged-1 plasmid & treated with pop culture reagent for 15 minutes. Strep-tag conjugated chimeric protein was purified using Strep·Tactin® resin. Purified truncated chimeric protein was resolved in 4-20% SDS-PAGE and protein expression was analyzed by Western blot using anti-StrepTag antibody.

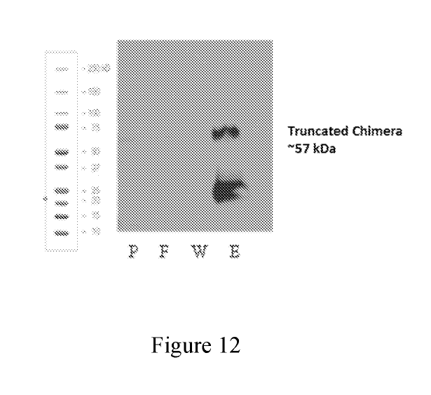

FIG. 13: PCR amplification of human truncated OX40L-Jagged-1-Fc. FIG. 13 is an agarose gel illustrating PCR amplified chimera fragment ran on 1% agarose gel at 100V for 30 minutes.

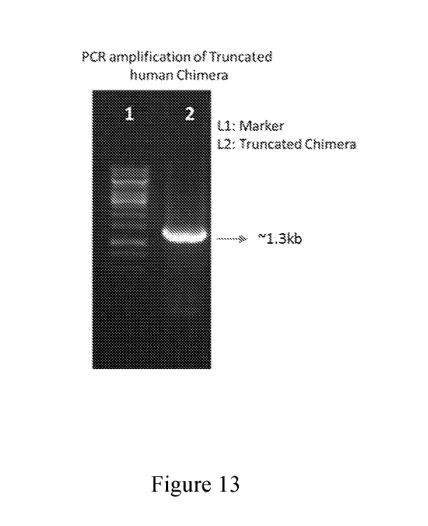

FIG. 14: Expression of truncated chimeric hOX40L-Jagged-1-Fc in 293T cells. FIG. 14 is a Western blot illustrating: Lanes: 1) Untransfected control, 2) full length chimeric hOX40L-Jagged-1-Fc, 3) truncated chimeric hOX40L-Jagged-1-Fc. Briefly, HEK 293T cells were transfected with the full-length and truncated chimera plasmids. After 48-72 hours, supernatants were incubated with protein A agarose and bound proteins purified. Purified proteins were resolved on SDS-PAGE and identified by western blot using anti-human IgG1 antibody. The expression level of the truncated construct was greater than the full-length construct.

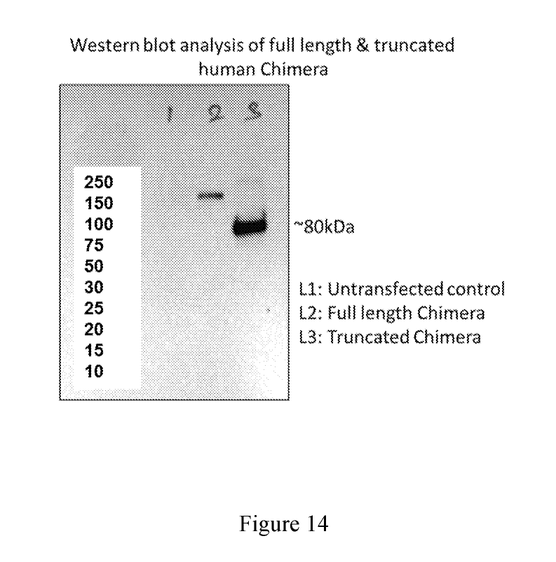

### DETAILED DESCRIPTION OF THE INVENTION

The invention provides chimeric polypeptides comprising a first and a second polypeptide and a linker wherein the first polypeptide is an OX40L polypeptide or fragment thereof and the second polypeptide is a Jagged-1 polypeptide or fragment thereof and methods of use. In particular embodiments the chimeric polypeptides disclosed herein can be used for treating an autoimmune patient.

Unless otherwise explained, all technical and scientific terms used herein have the same meaning as commonly understood by one of ordinary skill in the art to which a disclosed disclosure belongs.

The singular terms “a,” “an,” and “the” include plural referents unless context clearly indicates otherwise. Similarly, the word “or” is intended to include “and” unless the context clearly indicates otherwise.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

OX40L belongs to the tumor necrosis factor superfamily with co-stimulatory function. OX40L when expressed on antigen-presenting cells binds to OX40 expressed on T-cells.

The Jagged members (Jagged-1 and Jagged-2) of Notch family ligands have been shown to play important role in Treg expansion. Kared et al., 2006. Jagged2-expressing hematopoietic progenitors promote regulatory T cell expansion in the periphery through notch signaling. Immunity 25: 823-34; Hoyne et al., 2000. Serratel-induced notch signaling regulates the decision between immunity and tolerance made by peripheral CD4(+) T cells. Int Immunol 12: 177-85. The Notch family has 4 known receptors, Notch-1, -2, -3 and -4, and five known Notch ligands namely, DLL1, DLL3 and DLL4, and Jagged-1 and Jagged-2. Upon ligand binding, Notch receptors undergo two proteolytic cleavages. The first cleavage is catalysed by ADAM-family metalloproteases and is followed by the gamma-secretase mediated release of Notch intracellular domain (NICD). The NICD translocates to the nucleus where it forms a heterodimeric complex with various co-activator molecules and acts as a transcriptional activator. Fortini, 2009. Notch signaling: the core pathway and its posttranslational regulation. Dev Cell 16: 633-47. Expression of specific Notch ligands on dendritic cells (DCs) is known to activate specific T-cell responses. Minter et al., 2005. Inhibitors of gamma-secretase block in vivo and in vitro T helper type 1 polarization by preventing Notch upregulation of Tbx21. Nat Immunol 6: 680-8. While Jagged ligands have been shown to direct naive T-cells toward Th2 and/or Treg type of responses, Delta like ligands (DLL) have been shown to skew them towards a Th1 response. Amsen et al., 2004. Instruction of distinct CD4 T helper cell fates by different notch ligands on antigen-presenting cells. Cell 117: 515-26. Of relevance to the current invention are earlier reports of Treg expansion by hematopoietic progenitors expressing Jagged-2 and APCs over-expressing Jagged-1. Kared et al., 2006. Jagged2-expressing hematopoietic progenitors promote regulatory T cell expansion in the periphery through notch signaling. Immunity 25: 823-34; Hoyne et al., 2000. Serratel-induced notch signaling regulates the decision between immunity and tolerance made by peripheral CD4(+) T cells. Int Immunol 12: 177-85; Yvon et al., 2003. Overexpression of the Notch ligand, Jagged-1, induces alloantigen-specific human regulatory T cells. Blood 102: 3815-21; Vigouroux et al., 2003. Induction of antigen-specific regulatory T cells following overexpression of a Notch ligand by human B lymphocytes. J Virol 77: 10872-80. Similarly, DLL4 blockade ameliorated experimental autoimmune encephalomyelitis (EAE). Bassil et al., 2011. Notch ligand delta-like 4 blockade alleviates experimental autoimmune encephalomyelitis by promoting regulatory T cell development. J Immunol 187: 2322-8.

While OX40 is constitutively expressed on Tregs (Vu et al., 2007. OX40 co-stimulation turns off Foxp3+Tregs. Blood 110: 2501-10), Notch 3 is preferentially expressed on Tregs. Anastasi et al., 2003. Expression of activated Notch3 in transgenic mice enhances generation of T regulatory cells and protects against experimental autoimmune diabetes. J Immunol 171: 4504-11. In the context of TCR signaling, OX40 mediated-signaling can increase T cell proliferation by activating PI3 kinase (PI3K) and Akt, which are upstream activators of mTOR. Song et al., 2004. The co-stimulation-regulated duration of PKB activation controls T cell longevity. Nat Immunol 5: 150-8. GM-BMDCs derived from MHC class-II knockout mice were also able to expand Tregs and indicated that TCR signaling was not necessary. Bhattacharya et al., 2011. GM-CSF-induced, bone-marrow-derived dendritic cells can expand natural Tregs and induce adaptive Tregs by different mechanisms. Journal of leukocyte biology 89: 235-49. OX40 activation can form a signalosome consisting of CARMA1, PKC-Q and TRAF2 and cause enhanced NF-KB activation and contribute to cell survival and expansion. Rogers et al., 2001. OX40 promotes Bc1-xL and Bc1-2 expression and is essential for long-term survival of CD4 T cells. Immunity 15: 445-55; So et al., 2011. OX40 complexes with phosphoinositide 3-kinase and protein kinase B (PKB) to augment TCR-dependent PKB signaling. Journal of immunology 186: 3547-55. Notch 3 has been reported to activate both the alternate and the canonical NF-KB pathways. It can activate the alternative (Re1B) NF-KB pathway in murine thymocytes (Vacca et al., 2006. Notch3 and pre-TCR interaction unveils distinct NF-kappaB pathways in T-cell development and leukemia. EMBO J 25: 1000-8) via cytoplasmic IKKα and cooperate with canonical NF-KB in stimulating FoxP3 expression. Barbarulo et al., 2011. Notch3 and canonical NF-kappaB signaling pathways cooperatively regulate Foxp3 transcription. J Immunol 186: 6199-206. Thus NF-KB may be an important point of convergence between OX40 and Notch 3 signaling in Tregs.

Notch 1 has been reported to maintain expression of FoxP3 in peripheral Tregs in collaboration with TGFβ. Samon et al., 2008. Notch1 and TGF-beta 1 cooperatively regulate Foxp3 expression and the maintenance of peripheral regulatory T cells. Blood 112: 1813-21. Therefore, it is possible that different Notch paralogs can maintain FoxP3 expression depending on other signals and cellular context. It is well known that Foxp3⁺ Tregs are unable to proliferate or proliferate poorly when stimulated (Shevach et al., 2006. The lifestyle of naturally occurring CD4+ CD25+ Foxp3+ regulatory T cells. Immunological reviews 212: 60-73; Allan et al., 2005. The role of 2 FOXP3 isoforms in the generation of human CD4+ Tregs. The Journal of clinical investigation 115: 3276-84) and upon proliferation they lose Foxp3 expression. Notch 3 has been shown to co-operatively regulate Foxp3 expression through trans-activation of the Foxp3 promoter. Barbarulo et al., 2011. Notch3 and canonical NF-kappaB signaling pathways cooperatively regulate Foxp3 transcription. J Immunol 186: 6199-206. Therefore, it is likely that the interaction of Jagged-1 with Notch 3 helps sustain Foxp3 transcription while OX40 signalosome formation, in the absence of TCR signaling, may drive Foxp3⁺ Treg cell-proliferation. Thus, concurrent signals from Notch 3 and OX40 can allow Treg proliferation while sustaining Foxp3 expression.

The terms “T regulatory cell” or “Tregs” as used herein refer to a cell that can modulate a T cell response. Tregs express the transcription factor Foxp3, which is not upregulated upon T cell activation and discriminates Tregs from activated effector cells. Tregs are classified into natural or adaptive (induced) Tregs on the basis of their origin. Foxp3⁺ natural Tregs (nTregs) are generated in the thymus through MHC class II dependent T cell receptor. Adaptive Tregs are non-regulatory CD4+ T-cells which acquire CD25 (IL-2R alpha) expression outside of the thymus, and are typically induced by inflammation and disease processes, such as autoimmunity and cancer. The methods described herein can employ Tregs that expresses one or more of CD4, CD25 and Foxp3.

As used herein, the term “chimeric polypeptide” refers to a polypeptide consisting of one or more domains from different proteins. For example, the chimeric polypeptides disclosed herein comprise a first and a second polypeptide wherein one of the polypeptide is an OX40L polypeptide and one of the polypeptide is a Jagged-1 polypeptide. In one embodiment, the first polypeptide is a human OX40L polypeptide or fragment thereof (human OX40L amino acid sequence Uniprot ID: P23510 (SEQ ID NO: 1) and the second polypeptide is a human Jagged-1 polypeptide or fragment thereof (Human Jagged-1 amino acid sequence Uniprot ID: P78504 (SEQ ID NO: 5). In another embodiment, the first polypeptide is mouse OX40L polypeptide or fragment thereof (Mouse OX40L amino acid sequence Uniprot ID: P43488 (SEQ ID NO: 3) and the second polypeptide is a mouse Jagged-1 polypeptide or fragment thereof (Mouse Jagged1 amino acid sequence Uniprot ID: Q9QXX0 (SEQ ID NO: 7). In particular embodiments, the chimeric polypeptides disclosed herein comprise the extracellular domain of human OX40L or fragment thereof and the extracellular domain of human Jagged-1 or fragment thereof (SEQ ID NO: 9). In another embodiment, the chimeric polypeptides disclosed herein comprise the extracellular domain of mouse OX40L or fragment thereof and extracellular domain of mouse Jagged-1 or fragment thereof (SEQ ID NO: 11).

Additionally, the chimeric polypeptides disclosed herein include a linker joining the two polypeptides. The term “linker” is understood to mean a sequence of one or more amino acid residues which couple two proteins together. The polypeptide linker often is a series of amino acids of about 10-15 residues in length. In particular embodiments the linker of the chimeric protein is a polypeptide having at least about 90 or at least 95% identity to SEQ ID NO: 13 (DKTHTCPPCP) or SEQ ID NO: 42 (GCKPCICT). The linker allows for independent free movement of the extracellular domains of the OX40L and Jagged-1 proteins. In particular embodiments the chimeric protein comprises SEQ ID NO: 9 or SEQ ID NO: 11.

In particular embodiments the chimeric polypeptides disclosed herein comprise a Fc region of an immunoglobulin. The Fc region includes the CH2 and CH3 regions of the IgG heavy chain and the hinge region. The Fc chimeric protein is composed of the Fc domain of IgG genetically linked to the OX40L-Jagged-1 polypeptides. The use of the Fc domain is used to prolong the plasma half-life of the chimeric protein for use in improved therapeutic efficacy.

Derivatives and analogs of the chimeric polypeptides of the invention, are all contemplated, and can be made by altering their amino acid sequences by substitutions, additions, and/or deletions/truncations or by introducing chemical modifications that result in functionally equivalent molecules. It will be understood by one of ordinary skill in the art that certain amino acids in a sequence of any polypeptides may be substituted for other amino acids without adversely affecting the activity of the polypeptides.

The term “patient” as used herein refers to a mammal suffering from an autoimmune disease. In certain particular embodiments, the mammal is a human. In other certain embodiments, a patient is a human suffering from an autoimmune disease.

The term “autoimmune diseases” as used herein refers to a disease resulting from an immune response against a self-tissue or tissue component, including both self-antibody responses and cell-mediated responses. Exemplary autoimmune diseases that are suitable as targets for the inventive methods are type I diabetes mellitus (T1D), Crohn's disease, ulcerative colitis, myasthenia gravis, vitiligo, Graves' disease, Hashimoto's disease, Addison's disease and autoimmune gastritis and autoimmune hepatitis, rheumatoid disease, systemic lupus erythematosus, progressive systemic sclerosis and variants, polymyositis and dermatomyositis, pernicious anemia including some of autoimmune gastritis, primary biliary cirrhosis, autoimmune thrombocytopenia, Sjogren's syndrome, multiple sclerosis and psoriasis. One skilled in the art understands that the methods of the invention can be applied to these or other autoimmune diseases, as desired.

As used herein, the term “amount effective,” “effective amount” or a “therapeutically effective amount” refers to an amount of compound or composition sufficient to achieve the stated desired result, for example, treating or limiting development of autoimmune disease. The amount of the compound or composition which constitutes an “effective amount” or “therapeutically effective amount” may vary depending on the severity of the disease, the condition, weight, gender or age of the patient to be treated, the frequency of dosing, or the route of administration, but can be determined routinely by one of ordinary skill in the art. A clinician may titer the dosage or route of administration to obtain the optimal therapeutic effect.

In particular embodiments the autoimmune disease is an autoimmune thyroid disease (e.g., Grave's disease and Hashimoto disease). Autoimmune thyroid disease involves the dysfunction of the diseased thyroid gland and varies from hypothyroidism due to glandular destruction in Hashimoto's thyroiditis or blocking antibodies in primary myxedema to hyperthyroidism in Graves' disease due to thyroid simulating antibodies. In other particular aspects the autoimmune disease is Type 1 Diabetes Mellitus.

Cellular therapies for autoimmune diseases, including formulations and methods of administration are known in the art and can be applied to the T-regulatory cells and vectors described herein. See, for example, in EP1153131 A2, incorporated herein by reference.

Treat, treatment, treating, as used herein, means any of the following: the reduction in severity of an autoimmune disorder; the prophylaxis of one or more symptoms associated with an autoimmune disorder; the amelioration of one or more symptoms associated with an autoimmune disorder; the provision of beneficial effects to a subject with an autoimmune disorder, without necessarily curing the autoimmune disorder.

The chimeric polypeptides disclosed herein may be administered to a patient by any suitable means, directly (e.g., locally, as by injection, implantation or topical administration to a tissue locus) or systemically (e.g., parenterally or orally).

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used.

The foregoing may be better understood by reference to the following examples which are presented for purposes of illustration and are not intended to limit the scope of the invention.

### EXAMPLES

### Example 1

### Construction of Mouse OX40L-Jagged-1 Chimera

A cDNA coding mouse chimeric protein was produced comprising the extracellular domains of mouse OX40L and mouse Jagged-1. Mouse OX40L (Uniprot ID: P43488) also known as Tumor necrosis factor ligand superfamily member 4 (TNFSF4), is a 198 amino acids (aa) long protein (SEQ ID NO: 3). According to Uniprot protein repository (http://www.uniprot.org/uniprot/P43488), it is made up of three different domains: 1. Intracellular cytoplasmic domain (1-28 aa); 2. Transmembrane domain (29-50 aa); and 3. Extracellular domain (51-198 aa). Among these different domains, the extracellular domain binds with its cognate receptor OX40 expressed on target cells to transduce a signal. The 148 aa extracellular domain of OX40L which is coded by amino acids 51-198 for the construction was employed in the chimeric protein. Mouse OX40L nucleotide sequence is provided as SEQ ID NO: 4.

Mouse Jagged1 (JAG1, Uniprot ID: Q9QXX0), also called CD339, is an 1185 amino acids long protein (1218 aa with signal peptide) (SEQ ID NO: 7). According to Uniprot protein repository (http://www.uniprot.org/uniprot/Q9QXX0), it comprises of three different domains: 1. Extracellular cytoplasmic domain (34-1067 aa); 2. Transmembrane domain (1068-1093 aa); and 3. Intracellular domain (1094-1218 aa). Similar to OX40L, JAG1 also transmits its signal through binding of its extracellular domain to its cognate Notch family receptors expressed on target cells. The extracellular domain of JAG1 was employed in the chimeric protein. Mouse Jagged1 nucleotide sequence is provided as SEQ ID NO: 8.

The extracellular domains of mouse OX40L and mouse Jagged-1 were joined using a 8 amino acid linker sequence GCKPCICT (SEQ ID NO: 42) coding the hinge region present in mouse IgG1 Fc domain to enable flexible movement of the two proteins and to minimize/prevent protein-protein interaction. Additionally, a sequence coding for IL-2 signal sequence was added to the 5′ end and a mouse IgG1 Fc region was added to the 3′ end of the OX40L-Jagged-1 chimeric cDNA.

A commercially available pFUSE-mouse IgG1-Fc2 vector (Invivogen) designed for the construction of Fc-Fusion proteins was used to clone the chimeric OX40L-Jagged-1 cDNA. The Fc2 region of this vector contains the constant CH2 and CH3 domains of the IgG1 heavy chain and the hinge region. The hinge serves as a flexible spacer between the two partners of the chimeric protein. The linker used in the chimeric cDNA was specifically designed to allow independent free movement of the extracellular domains of the OX40L and Jagged-1 proteins. Furthermore, presence of IgG1 tag provided for easy purification of Fc-Fusion proteins by single-step protein A/G affinity chromatography. The pFUSE-mouse IgG1-Fc2 vector contains IL-2 signaling sequence (IL-2ss) to facilitate efficient secretion of Fc-fusion proteins so that proteins can be easily purified from cell culture supernatant in its native state to ensure retention of their biological activities.

The PCR strategy employed for the amplification of the chimeric nucleic acid sequences was as follows:
1. Amplification of nucleotide sequence coding for the extracellular domain of OX40L with a 3′ Fc linker overhang used: a mouse OX40L cDNA clone as template (cDNA that we cloned from mouse bone marrow dendritic cells), Sense primer Fc-OX40L-ecto-F (5′-GCG CGA ATT CGC AAC TCT CTT CCT CTC CGG CA-3′; SEQ ID NO: 14) and anti-sense primer pFUSE-OX40L-Linker-R (5′-TGT ACA TAT GCA AGG CTT ACA ACC CAG TGG TAC TTG GTT CAC AGT -3′; SEQ ID NO: 15). PCR conditions were as follows: 1. Initial denaturation at 95° C. for 5 min, 2. Denaturation at 95° C. for 30s, 3. Annealing at 48-68° C. (gradient) for 30s, 4. Extension at 72° C. for 30s for 35 cycles (Slides 1&2; scheme and figures for PCR). This generated an OX40L-specific PCR product with a 5′ Eco R1 restriction site (˜480 bp) (FIGS.-1 and -2; scheme and figures for PCR respectively).
2. Amplification of nucleotide sequence coding for the extracellular domain of JAG1 with Fc linker overhang (complementary to overhang amplified with OX40L) at 5′ end used: a mouse JAG1 specific DNA clone as template (Accession # BC058675), sense primer Fc-linker-JAG1-ecto-F (5′-GGT TGT AAG CCT TGC ATA TGT ACA CAG TTT GAG CTG GAG ATC CTG TCC-3′; SEQ ID NO: 16) and anti-sense primer Fc-JAG1-ecto R (5′-GCG CGA ATT CCC ATC TGT TCT GTT TTT CAG AGG ACG-3′; SEQ ID NO: 17). PCR conditions were as follows: 1. Initial denaturation at 95° C. for 5 min, 2. Denaturation at 95° C. for 1 min, 3. Annealing at 48-68° C. for 1 min (gradient), 4. Extension at 72° C. for 3 min for 35 cycles. This generated a JAG1 specific PCR product with a 3′ Eco R1 restriction site (˜3.1 kB) (FIGS.-1 and -2; scheme and figures for PCR respectively).
3. The OX40L and Jagged-1 PCR products from steps 1 & 2 were mixed in equimolar ratio as templates and the chimeric OX40L-Jagged-1 fragment was amplified by overlap extension PCR, (whereby the two PCR products anneal through the short complementary hinge region segment (24 nucleotides) common to both) using the following primers:OX40L sense primer Fc-OX40L-ecto-F (5′-GCG CGA ATT CGC AAC TCT CTT CCT CTC CGG CA-3′; SEQ ID NO: 18) and JAG1 anti-sense primer Fc-JAG1-ecto R (5′-GCG CGA ATT CCC ATC TGT TCT GTT TTT CAG AGG ACG-3′; SEQ ID NO: 19). PCR conditions were as follows: 1. Initial denaturation at 95° C. for 5 min, 2. Denaturation at 95° C. for 1 min, 3. Annealing at 48-68° C. for 1 min, 4. Extension at 72° C. for 3 min for 35 cycles. This generated a chimeric OX40L-Jagged-1 fragment ('3.5 kb) (Slides 1&2; scheme and figures for PCR respectively).
4. The PCR amplified chimeric OX40L-Jagged-1 fragment and pFUSE-mouse IgG1-Fc2 vector were digested with restriction enzyme Eco R1, ligated with Quick ligase and transformed in to DH5-α bacteria. Chimera clones were selected using ampicillin selection (100 μg/ml). pFUSE-chimera plasmid was isolated from cultures of selected E. coli clones.
5. Orientation and reading frame of the chimera sequence was analyzed by restriction digestion using enzymes Eco R1 and Bg1II. There are two Bg1 II sites within the JAG1 ectodomain sequence and none in OX40L ectodomain. Eco R1 releases the inserted chimeric fragment. However, digestion with Bg1 II yields the following fragments: ˜500 bp for OX40L PCR product (which was separately cloned as a control); ˜800 bp+˜2.2 kb for JAG1 PCR product (which was separately cloned as a control); and ˜1.3 kb+2.2 kb for chimeric OX40L-JAG1 product and A 4.2 kb product for the vector. The cloned plasmid constructs released the expected DNA bands confirming the respective clones (FIG.-3). These clones were further confirmed by Sanger sequencing.

For bacterial expression, the cloned pFUSE-chimera plasmid was used as a template to amplify chimeric OX40L-Jagged-1 PCR product using forward primer pet15b-OX40L-F (5′-GCG CCA TAT GCA ACT CTC TTC CTC TCC GGC A-3′; SEQ ID NO: 20) and one of the following two reverse primers pet15b-Fc-R (5′-GCG CGG ATC CTC ATT TAC CAG GAG AGT G-3′; SEQ ID NO: 21) pet15b-JAG1-R (5′-GCG CGG ATC CTC AAT CTG TTC TGT TTT TCAG AGG ACG-3′; SEQ ID NO: 22) for expressing chimeric OX40L-Jagged-1 with and without a C-terminal Fc tag respectively. The PCR chimeric OX40L-Jagged-1 products and the pET15b plasmid were digested with restriction enzymes Nde 1 and BamH 1, ligated and used to transform E. coli DH5-α cells. Recombinant pET15b-chimera clones were selected on LB agar plates containing ampicillin.

For expression of chimeric OX40L-Jagged-1 in insect cells using baculoviral expression system, the chimeric OX40L-Jagged-1 construct was PCR amplified using pFUSE-chimera plasmid as a template with the forward primer Bacu-OX40L-F (5′-CGC GGG ATC CAC CAT GCA ACT CTC TTC CTC TCC GGC A-3′; SEQ ID NO: 23) and the Bacu-Reverse primer (5′-CGC GGC GGC CGC CCA GCT AGC GAC ACT GGG ATC-3′; SEQ ID NO: 24). The PCR product and plasmid pFastBac 1 (Life technologies) were digested with restriction enzymes BamH 1 and Not 1, ligated and used to transform E. coli DH5-α cells. Recombinant pFastBac1-chimera clones were selected on LB agar plates containing ampicillin. Clones were confirmed by restriction digestion and used to isolate plasmids. Cloned plasmids were used to further transform E. coli DH10Bac cells (Life Technologies) to generate recombinant Bacmids for generation of Baculovirus. Recombinant Bacmids were selected on LB agar plates containing gentamycin, kanamycin, tetracyclin, X-gal and IPTG according to standard protocol (Life technologies). Selected clones were used to isolate recombinant chimera-Bacmids and confirmed by PCR.

### Example 2

### Expression and Purification of Mouse OX40L-JAG1-Fc Chimera

Recombinant pET15b-chimera plasmids were used to transform E. coli BL21 cells for bacterial expression. Clones were inoculated in LB broth and growth overnight in the presence of ampicillin. Overnight cultures were used to inoculate fresh LB broth in the morning and grown at 37° C. with constant shaking at 220 rpm for 2-3 hours (until cultures reached an OD of 0.4-0.6). Protein expression was induced with 1 mM IPTG. Cells were harvested after every hour post induction for a period up to 4 hours. Harvested cells were lysed by boiling and lysate was resolved on SDA-PAGE. Protein expression was analyzed by staining with coomassie blue and by western blot using anti-mouse IgG1 antibodies. No chimeric protein expression was detected by either method.

Bacmid-chimera was used to transfect SF21 insect cells using cellfectin (Life Technologies) and grown on SF-900 media (Life Technologies). After 72 hours, media supernatant containing recombinant Baculovirus was harvested and used to infect adherent SF21 cells. After 72 hours, cells were harvested, lysed and resolved on SDS-PAGE. Expression of chimeric OX40L-Jagged-1 protein was analyzed by Coomassie Blue and western blot using anti-mouse IgG1 antibodies. No protein was detected by either method.

Recombinant pFUSE-chimera plasmids (3 clones, shown in lanes 4-6 in FIG.-4) were used to transfect HEK 293 cells using lipofectamine (Life Technologies). Control clones for OX40L-Fc expression (shown in lane-1 in FIG.-4) and Jag1-Fc expression (2 clones shown in lanes 2 and 3 in FIG.-4) were also used side by side for comparison. 72 h post-transfection, chimeric protein secreted from HEK 293 cells were purified from culture supernatant using protein A/G beads by IgG affinity purification. Purified protein were resolved on SDS-PAGE and analyzed by western blot using anti-mouse IgG antibodies. Western Blot revealed the expression of chimeric OX40L-Jagged-1 at approximately 160 kDa FIG.-4).

Recombinant pFUSE-chimera plasmid was also used to transfect CHO K1 cells. Stable chimera producing clones were selected in the presence of Zeocin. These stably expressing cells were further cloned and individual clones screened by Flow cytometry based analysis for intracellular expression of chimeric OX40L-Jagged-1 using PE labelled anti mouse IgG specific antibody. Clone F9 was selected as a high expressing clone (˜90% positive for expression of chimeric OX40L-Jagged-1) (FIG.-5).

The gel band on SDS-PAGE corresponding to the molecular weight of chimeric OX40L-Jag1-Fc protein was excised as determined by western blot (˜160 kDa as shown in FIG.-4). Chimeric protein bands resolved in gels were excised and washed in 50% acetonitrile, reduced of sulfide bonds in 60 mM DTT, alkylated of free sulfhydryl groups in iodoacetamide, 50 mM ammonium bicarbonate (pH 8.0) and 5 mM EDTA, and then incubated in trypsin [in 50 mM ammonium bicarbonate (pH 8.0) solution overnight. The tryptic peptides were injected onto a reversed phase column (75 um×150 mm Zorbax SB300 C-18, Agilent Technologies) connected to a Dionex Ultimate 3000 two dimensional microcapillary HPLC system and a Thermo Orbitrap Velos Pro mass spectrometer equipped with an nanospray interface. The samples were chromatographed using a binary solvent system consisting of A: 0.1% formic acid and 5% acetonitrile and B: 0.1% formic acid and 95% acetonitrile at a flow rate of 250 nL/min. A gradient was run from 15% B to 45% B over 60 minutes. The mass spectrometer was operated in positive ion mode with the trap set to data dependent MS/MS acquisition mode. The instrument was set to complete a mass scan from 400-1800 daltons in one second. Peaks eluting from the LC column that have ions above 25,000 arbitrary intensity units trigger the ion trap to isolate the ion and perform an MS/MS experiment scan after the MS full scan. Data files created were then processed using Thermo Xcalibur software to produce an intermediate file containing the peaks detected and fragmented. These intermediate files were transferred to a sequence database searching server MASCOT (http://www.matrixscience.com) to search and align with known protein sequence. Our MS analysis results identified the presence of 2 mouse IgG1 specific signature peptides such as DVLTITLTP (SEQ ID NO: 25) and NTQPIMDTDGSYFVYSK (SEQ ID NO: 26) thus confirming the presence of the fusion protein.

### Example 3

### Mouse Treg Expansion by Mouse Chimeric OX40L-JAGGED-1-Fc

Protein A/G affinity purified mouse chimeric OX40L-Jagged-1-Fc was dialyzed and concentrated. CD4+ T-cells were isolated from spleens of non-obese diabetic (NOD) mice using CD4+ T-cell isolation kit (Miltenyi). Purified CD4+ T-cells were labelled with cell proliferation dye (Cell trace—Violet), mixed with splenic antigen presenting cells and incubated in RPMI 1640 medium (10% FBS) for 5 days in the presence of chimeric OX40L-Jagged-1-Fc and IL-2. OX40L-Fc alone, expressed and purified by similar methods was also used as a control. After 5 days of culture, cells were fixed, permeabilized, stained for CD4 and FoxP3, and analyzed for cell proliferation by FACS. Cell proliferation was measured by Cell trace violet dilution. While control FoxP3+ cells (un-supplemented) showed minimal proliferation (˜3.6%), cells supplemented with OX40L-Jagged-1-Fc alone showed appreciable proliferation (˜28%) over OX40L alone (˜10%) which further increased upon addition of IL-2 (˜40%). This indicated that the chimeric OX40L-Jagged-1 was functionally active and capable of Treg proliferation (FIG.-6).

### Example 4

### Construction of Human Chimera

A human chimeric protein was constructed comprising OX40L-Jagged-1 extracellular domains fused to a human IgG1-Fc2. The chimeric construct was designed to contain the indicated sub-parts in the following order from N- to C-terminus: IL-2 signal sequence, extracellular domain of OX40L-Hinge region of human IgG1-Fc and the extracellular domain of Jagged-1.

Human OX40L (Uniprot ID: P23510), also known as Tumor necrosis factor ligand superfamily member 4, is a 20 kDa membrane protein encoded by 183 amino acids (aa) (SEQ ID NO: 1). According to Uniprot protein repository (http://www.uniprot.org/uniprot/P23510), it is made up of three different domains: 1. Intracellular cytoplasmic domain (1-23 aa); 2. Transmembrane domain (24-50 aa); and 3. Extracellular domain (51-183 aa). Among these different domains, extracellular domain binds to its cognate receptor OX40 expressed on target cells to transduce signal. Therefore, soluble form of OX40L extracellular domain should be able to bind to its receptor OX40 to transduce signal. Hence, the extracellular domain of OX40L which consists of amino acids 51-183 was selected for the chimeric protein. Human OX40L nucleotide sequence is provided as SEQ ID NO: 2.

Human Jagged-1 (Jag1, Uniprot ID: P78504), also known as CD339, is a 135 kDa membrane protein encoded by 1218 amino acids (SEQ ID NO: 5). According to Uniprot protein repository (http://www.uniprot.org/uniprot/P78504), it comprises of three different domains: 1. Extracellular cytoplasmic domain (34-1067 aa); 2. Transmembrane domain (1068-1093 aa); and 3. Intracellular domain (1094-1218 aa). Similar to OX40L, JAG1 also transmits its signal through binding of its extracellular domain with the cognate Notch family receptors expressed on target cells. Human Jagged-1 nucleotide sequence is provided as SEQ ID NO: 6.

Through an Expasy bioinformatics tool Protparam (http://web.expasy.org/cgi-bin/protparam/), the stability index of the Fc linked OX40L-JAG1 chimera was calculated and a 10 aa (DKTHTCPPCP; SEQ ID NO: 13) stable linker from human immunoglobulin G1 hinge region was selected as the linker for the chimeric protein. Existence of the linker region provides for free movement of each component of the chimeric protein without hindering their ability to bind to their corresponding receptors and mediate signaling.

A commercially available pFUSE-human IgG1-Fc2 vector (Invivogen) designed for the construction of Fc-Fusion proteins was used. The Fc2 region of the vector contains the constant CH2 and CH3 domains of the IgG1 heavy chain and the hinge region. The Fc2 has relatively low effector activities such as antibody dependent cell mediated cytotoxicity and complement dependent cell cytotoxicity and therefore, most suitable for therapeutic applications. The selection of the hinge region was critical as it serves as a flexible spacer between the two partners of the chimeric Fc-fusion protein. This flexibility afforded by the spacing is critical because it can minimize or prevent protein-protein interaction, allow for free spatial movement of the extracellular domains of OX40L and Jagged-1 proteins and thus help maintain their three dimensional structure required for their biological function. Furthermore, presence of IgG1-Fc2 tag allowed for easy purification of Fc-Fusion chimeric protein in a single-step protein A or protein G affinity chromatography. The vector contains IL-2 signal sequence (IL-2ss), which facilitates efficient secretion of Fc-fusion proteins so that proteins can be easily purified from cell culture supernatant; ensuring retention of their native structure required for their biological activity.

The PCR strategy employed for the amplification of the chimeric nucleic acid sequences was as follows:
1. Amplification of nucleotide sequence coding for the extracellular domain of OX40L with a Fc linker sequence overhang at 3′ end: For this amplification a human OX40L cDNA clone was used as the template (Clone ID: 4510740), along with the sense primer 5′ TAA GGA ATC CGCT CCA CTG TGT CGG GGA CAC C 3′ (SEQ ID NO: 27) and the anti-sense primer 5′-TGG GCA CGG TGG GCA TGT GTG AGT TTT GTC CGC ACG GCC CCC GGG GAC CTC CA 3′(SEQ ID NO: 28). PCR condition was as follows: 1) Initial denaturation at 95° C. for 5 min, 2) Denaturation at 95° C. for 30s, 3) Annealing at 50° C. for 30s, 4) Extension at 72° C. for 30s for 35 cycles (FIG.-7A).
2. Amplification of nucleotide sequence coding for the extracellular domain of Jag1 with a 5′ end Fc linker overhang (complementary to overhang amplified with OX40L): For this amplification, a Jagged-1 cDNA clone was used as template (Clone ID: 8991923), along with the sense primer 5′-CAG TTC GAG TTG GAG ATC CTG TCG ACA AAA CTC ACA CAT GCC CAC CGT GCC CA-3′ (SEQ ID NO: 29) and the anti-sense primer 5′ TGC TGA TAT CCC ATC TGT TCT GTT CTT CAG AGG CC 3′ (SEQ ID NO: 30). PCR condition was as follows: 1) Initial denaturation at 95° C. for 5 min, 2) Denaturation at 95° C. for 1 min, 3) Annealing at 50° C. for 1 min, 4) Extension at 72° C. for 3 min for 35 cycles (FIG.-7B).
3. Assembly linker PCR using OX40L-Linker and JAG1-Linker PCR products as templates using OX40L sense primer 5′ TAA GGA ATC CGCT CCA CTG TGT CGG GGA CAC C 3′ (SEQ ID NO: 27) and JAG1 anti-sense primer 5′ TGC TGA TAT CCC ATC TGT TCT GTT CTT CAG AGG CC 3′ (SEQ ID NO: 30). Molar ratio of the templates was calculated based on the stoichiometry between OX40L and Jag1 linker PCR product sizes. Optimal amplification attained when OX40L: JAG1 linker template were mixed at a ratio of 1:5. PCR condition was as follows: 1) Initial denaturation at 95° C. for 5 min, 2) Denaturation at 95° C. for 1 min, 3) Annealing at 50° C. for 1 min, 4) Extension at 72° C. for 3 min for 35 cycles (FIG.-1C).

PCR amplified chimera fragment was resolved in a 1% agarose gel at 100V for 30 minutes and was purified from the gel. Chimera fragment and pFUSE-human IgG1-Fc2 vectors were digested with restriction enzyme EcoRV for 2 h at 37° C. Digested DNA fragments were resolved in a 1% agarose gel at 100V for 30 minutes and then purified from the gel. After purification, digested chimera fragment and pFUSE-human IgG1-Fc vectors were ligated with Quick ligase at a molar ratio of 5:1 at room temperature for 30 min. Ligated pFUSE-human chimera cDNA was transformed into DH5-α bacteria. Chimera clones were selected by ampicillin selection (100 μg/ml). PFUSE-Chimera plasmid was purified from E. coli. Orientation and reading frame of the chimera sequence was confirmed by Sanger DNA sequencing.

For bacterial expression, the cloned pFUSE-human OX40L-JAG1-Fc chimera plasmid was used as a template to amplify chimeric OX40L-Jag1 PCR product using forward primer pet15b-OX40L-F (5′-ACT TCA TAT GAT GGT ATC ACA TCG GTA TCC TCG AAT-3′; SEQ ID NO: 31) and one of the following two reverse primers pet15b-Fc-R (5′-CTA GGG ATC CTT ATC ATT TAC CCG GAG ACA GGG AGA GG-3′; SEQ ID NO: 32) pet15b-JAG1-R (5′-CTA GGG ATC CTT AAT CTG TTC TGT TCT TCA GAG GCC G-3′; SEQ ID NO: 33) for expressing chimeric OX40L-Jag1 with or without a C-terminal Fc tag respectively. The PCR chimeric OX40L-JAG1 products and the pET15b plasmid were digested with restriction enzymes Nde 1 and BamH 1, ligated and used to transform E. coli DH5α cells. Recombinant pET15b-chimera clones were selected on LB agar plates containing ampicillin.

For expression of chimeric OX40L-JAG1-Fc chimera in insect cells using baculoviral expression system, the cloned pFUSE-human OX40L-JAG1-Fc chimera was PCR amplified using pFUSE-chimera plasmid as a template with forward primer 5′-ACT TC TCG AGAC CATG TAC AGG ATG CAA CTC CTG TCT TGC AT-3′ (SEQ ID NO: 34) and 5′ CTA GAAA GCT TT CAT TTA CCC GGA GAC AGG GAG AGG CTC 3′ (SEQ ID NO: 35). The PCR product and plasmid pFastBac1 (Life technologies) were digested with restriction enzymes XhoI and KpnI, ligated and used to transform E. coli DH5α cells. Recombinant pFastBac1-chimera clones were selected on LB agar plates containing ampicillin. Clones were confirmed by restriction digestion. Cloned plasmids were used to further transform E. coli DH10Bac cells (Life Technologies) to generate recombinant Bacmids for the generation of Baculovirus. Recombinant Bacmids were selected on LB agar plates containing gentamycin, kanamycin, tetracyclin, X-gal and IPTG according to standard protocol (Life technologies). Selected clones were used to isolate recombinant chimera-Bacmids and were confirmed by PCR.

### Example 5

### Expression and Purification of Human OX40L-JAG1-Fc Chimera

Recombinant pET15b-chimera plasmids were used to transform E. coli BL21 cells for bacterial expression. Clones were inoculated in LB broth and growth overnight in the presence of ampicillin. Overnight cultures were used to inoculate fresh LB broth in the morning and grown at 37° C. with constant shaking at 220 rpm for 2-3 hours (until cultures reached an OD of 0.4-0.6. These cultures were then treated with 1 mM IPTG to induced protein expression. Cells were harvested after every hour post induction for a period up to 4 hours. Harvested cells were lysed by boiling and lysate resolved on SDA-PAGE. Protein expression was analyzed by staining with coomassie blue and by western blot using anti-human IgG1 antibodies. No chimeric protein expression was detected when either clone (with or without Fc tag) was used for bacterial transformation.

Bacmid-chimera was used to transfect SF21 insect cells using cellfectin (Life Technologies) and grown on SF-900 media (Life Technologies). After 72 hours, media supernatant containing recombinant Baculovirus was harvested and used to infect adherent SF21 cells. After 72 hours, cells were harvested, lysed and resolved on SDS-PAGE. Expression of chimeric OX40L-Jag1 protein was analyzed by Coomassie Blue and western blot using anti-mouse IgG1 antibodies. No chimeric protein expression was detected by either method.

Different mammalian cell lines were screened such as, CHO (Chinese Hamster Ovary) cells, HEK293 (Human Embryonic Kidney epithelial cells) and HEK293T cells for the optimal production of the chimeric protein. Transfection conditions were optimized with different concentrations of plasmid DNA and transfection reagent. Optimal chimera expression was observed with HEK293T cells. Therefore, for further protein chimeric protein production HEK293T cells were used: 1×10⁶ HEK293T cells were transfected with 2 μg of purified pFUSE-Chimera plasmid DNA. 72 h Post-transfection, Chimeric protein secreted from HEK293T cells was purified from culture supernatant using protein-A beads. Subsequently, a kill curve experiment was performed to determine the optimal antibiotic (Zeocin) concentration at which un-transfected HEK-293T cells died after 10 days of selection. Based on this, stable chimera producing clones were selected by Zeocin selection (200 μg/ml) and screened by Flow cytometry (FIG.-8A) and Western blot using human IgG1 specific antibody (FIG.-8B). For large scale protein production cell clones selected for higher expression were cultured in DMEM-F12 media supplemented with 5% FBS and penicillin/streptomycin. For large scale protein production, Cell culture supernatants were incubated with protein-A agarose beads overnight at 4° C. Presence human IgG1-Fc tag in chimeric protein enabled binding of chimeric protein to protein-A. Beads were washed with 1X Phosphate Buffered Saline (PBS) to remove non-specifically bound proteins. Chimeric protein was eluted using an acidic elution buffer containing sodium citrate (pH 3.0) and immediately neutralized with basic neutralization buffer containing TRIS (pH 9.0). Purified protein was dialyzed against PBS and filter sterilized by passing it through a 0.22 μ filter. Purified protein was stored at −70° C. for further use.

Purified chimeric protein was resolved in 4-20% SDS-PAGE (FIG.-8B shown by arrow). Chimeric protein bands resolved in gels were excised and washed in 50% acetonitrile, reduced of sulfide bonds in 60 mQM DTT, alkylated of free sulfhydryl groups in iodoacetamide, 50 mM ammonium bicarbonate (pH 8.0) and 5 mM EDTA, and then incubated in trypsin [in 50 mM ammonium bicarbonate (pH 8.0) solution overnight. The tryptic peptides were injected onto a reversed phase column (75 um×150 mm Zorbax SB300 C-18, Agilent Technologies) connected to a Dionex Ultimate 3000 two dimensional microcapillary HPLC system and a Thermo Orbitrap Velos Pro mass spectrometer equipped with an nanospray interface. The samples were chromatographed using a binary solvent system consisting of A: 0.1% formic acid and 5% acetonitrile and B: 0.1% formic acid and 95% acetonitrile at a flow rate of 250 nL/min. A gradient was run from 15% B to 45% B over 60 minutes. The mass spectrometer was operated in positive ion mode with the trap set to data dependent MS/MS acquisition mode. The instrument was set to complete a mass scan from 400-1800 daltons in one second. Peaks eluting from the LC column that have ions above 25,000 arbitrary intensity units trigger the ion trap to isolate the ion and perform an MS/MS experiment scan after the MS full scan. Data files created were then processed using Thermo Xcalibur software to produce an intermediate file containing the peaks detected and fragmented. The intermediate files were transferred to a sequence database searching server MASCOT (http://www.matrixscience.com) to search and align with known protein sequence. The MS analysis results identified the presence of four human JAG1 specific signature peptides such as VTAGGPCSFGSGSTPVIGGNTFNLK (SEQ ID NO: 36), NTGVAHFEYQIR (SEQ ID NO: 37), DLVNDFYCDCK (SEQ ID NO: 38), and EMMSPGLTTEHICSELR (SEQ ID NO: 39) and, two human IgG1-Fc specific signature peptides TPEVTCVVVDVSHEDPEVKFNW (SEQ ID NO: 40) and YVDGVEVHNAK (SEQ ID NO: 41). Thus, the presence of human OX40L-JAG1-Fc chimera was confirmed by Western blot and HPLC-MS.

### Example 6

### Human Treg Expansion Induced by Human OX40L-JAG1-Fc Chimera

Human CD4+T-cells isolated from peripheral blood mononuclear cells were stained with proliferation marker (Cell trace—Violet), treated with chimera (5□g /ml) and IL-2 (10 IU/ml) and cultured in a 5% CO₂ incubator at 37° C. for 5 days. After 5 days of culture, cells were fixed, permeabilized and stained with CD4-APC, CD25-PE and FOXP3-FITC. CD4+ and CD4+ CD25+T-cells were gated and proliferation of CD4+FOXP3 and CD4+CD25+FOXP3+ Treg cells was measured by Cell trace violet dilution. Results are expressed as percentages of resting and proliferating Treg cells (FIG.-9). Values in the upper right and left quadrants represent percentage of resting and proliferating Treg cells respectively. The results showed a 4 fold highly significant (p<0.01, n=3) increase in FOXP3+ Treg proliferation in human chimera and IL-2 co-treated cells when compared with control cells treated with IL-2 alone.

### Example 7

### Production and Expression of a Truncated Mouse Chimeric OX40L-Jagged-1-Fc Protein

A truncated mouse chimeric OX40L-Jagged-1-Fc construct was produced comprising the complete 148 amino acid extracellular domain of mouse OX40L (coded by amino acids 51-198 of Uniprot ID: P43488) and a truncated Jagged-1 ectodomain (containing DSL domain and EGF like repeats 1-3 spanning 34-334 amino acids of Q9QXX0) linked by a hinge region derived from mouse IgG1 Fc.

Mouse Jagged-1 ectodomain is 1034 amino acids long (coded by amino acids 34-1067 of Q9QXX0), however, only the DSL domain (amino acids 185-229) is considered indispensable for the interaction of Jagged-1 with Notch receptors and the first two. EGF-like repeats (amino acids 230-263 and 264-294 respectively) are likely helpful to improve the affinity of the ligand-receptor interaction. The other EGF-like repeats do not play a significant role in regulation of the binding of Jagged-1 with Notch receptors (Shimizu et al. Mouse jagged1 physically interacts with notch2 and other notch receptors. Assessment by quantitative methods. J Biol Chem. 1999 12; 274(46):32961-9).

### Cloning of Mouse Truncated OX40L-Jagged-1-Fc Chimeric Protein

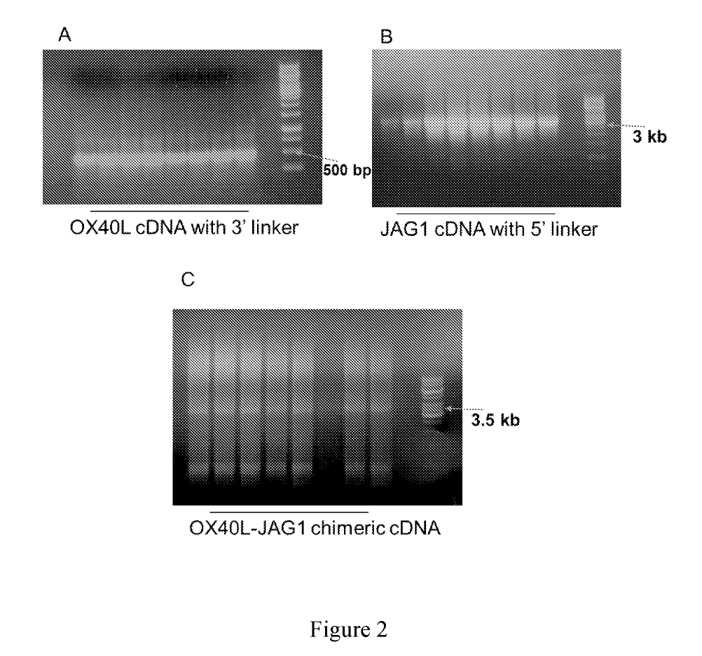

PCR amplification of the truncated chimeric DNA fragment was accomplished using sense primer; 5 GCGCGATATCGCAACTCTCTTCCTCTCCGGCA3′ (SEQ ID NO: 43) and anti-sense primer 5′GCGCCCATGGCTTCACAGTTGGGGCCCGAG3′ (SEQ ID NO: 44). Underlined sequences indicate EcoRI and Bg1II sites respectively. Plasmid DNA of full length mouse chimera (pFUSE-mIgG1-Fc2 containing full length mOX40L-JAG1 chimeric insert as described above) was used as template for the PCR amplification. PCR conditions were as follows: 1) Initial denaturation at 95° C. for 5 min, 2) Denaturation at 95° C. for 30s, 3) Annealing at 50° C. for 30s, 4) Extension at 72° C. for 30s for 35 cycles. PCR amplified cDNA for truncated chimera was resolved on a 1% agarose gel, purified using commercial kits and digested with restriction enzymes EcoR1 and Bg1II. The plasmid pFUSE-mIgG1-Fc2 vector was also digested with the same set of restriction enzymes (plasmid restriction map shown in FIG. 2). Restricted DNA fragments (both PCR product of truncated chimera and plasmid) were resolved again on 1% agarose gel and gel purified. After purification, digested chimera fragment and pFUSE-mIgG1-Fc vectors were ligated with Quick ligase at a molar ration of 3:1 at room temperature for 20 min. Ligated pFUSE-mouse chimera was transformed in to DH5-α bacteria. Chimera clones were selected by ampicillin selection (100 μg/ml). PFUSE-Chimera plasmid was purified from E. coli. Orientation and reading frame of the chimera sequence was confirmed by Sanger DNA sequencing.

### Expression and Purification of Truncated Mouse Chimeric Protein in HEK-293 Cells

Expression of mouse full-length and truncated mOX40L-Jagged-1-Fc chimeric proteins in HEK293T cells was accomplished as follows: 1×10⁶ HEK-293 cells were transfected with 2 μg of purified pFUSE-plasmid DNAs (containing cDNAs of OX40L, full length chimeric mOX40L-Jagged-1-Fc and 3 independent clones of truncated mOX40L-Jagged-1-Fc numbered 1, 2 and 3). Cells were cultured in DMEM-F12 media supplemented with 10% FBS. 48-72 h after transfection, proteins secreted from HEK-293 cells were isolated from culture supernatant by affinity purification using protein A/G-agarose beads. In brief, cell culture supernatants were incubated with protein-A/G agarose beads overnight at 4° C. Bound proteins were eluted by acidic elution buffer (pH 3.0) and immediately neutralized with basic neutralization buffer (pH 9.0). Purified chimeric protein was resolved in 4-20% SDS-PAGE and comparison of expression/purification was done by Western blot using anti-mouse IgG1 antibody (FIG.-10). Comparative analysis showed significantly increased yield for truncated chimera compared to full length chimera.

### Example 8

### Cloning and Expression of Mouse Truncated Chimeric Protein in Insect Cells

The same truncated chimeric protein as described above was used in an insect cell expression system, InsectDirect System (EMD, Novagen).

InsectDirect system utilizes a ligation-independent cloning (LIC) vector which enables directional cloning of PCR products without the need for restriction enzyme digestion or ligation reactions. The LIC method uses the 3′ to 5′ exonuclease activity of T4 DNA Polymerase to create specific 13- or 14-base single stranded overhangs in the Ek/LIC vector. PCR products with complementary overhangs are created by building appropriate 5′ extensions into the primers. Therefore, cDNA of mouse truncated OX40L-Jagged-1-Fc chimera was PCR-amplified using the following sense and anti-sense primers 5′ GAC GAC GAC AAG ATG caa ctc tct tcc tct ccg gca-3′ (SEQ ID NO: 45) and 5′ GA GGA GAA GCC CGG ttc aca gtt ggg gcc cga gta-3′(SEQ ID NO: 46) respectively. Underlined sequences are overhangs which will ligate to the complementary overhangs in the vector. PCR condition was as follows: 1) polymerase activation at 95 ° C. for 2 min; 2) denaturation at 95 ° C. for 20s; 3) annealing at 50° C. for 10s; 4) extension 70° C. for 15s and for 20 cycles. PCR products were cleaned up to remove residual dNTPs and DNA polymerase. Purified PCR product was treated with LIC-qualified T4 DNA Polymerase in the presence of dATP to generate specific vector-compatible overhangs. Annealing of pIEx-10-Ek/LIC vector DNA and OX40L- JAG1 chimeric insert DNA was done as follows: In a sterile 1.5-ml microcentrifuge tube 1 μl Ek/LIC Vector and 2 μl T4 DNA Polymerase treated Ek/LIC insert (0.02 pmol) were added and incubated at 22° C. for 5 min. Later, 1μl of 25 mM EDTA was added to a total volume of 4 μl. Mixed by stirring with pipet tip and incubated at 22° C. for 5 min. Resulting DNA products were transformed in to E. coli NovaBlue GigaSingles™ Competent Cells. Resulting colonies were screened for inserts by colony PCR using pIEx-10-Ek/LIC vector-specific primers, followed by agarose gel electrophoresis (FIG.-11). After identifying positive clones, plasmid DNA were isolated from bacteria and subjected to Sanger sequencing analysis.

For protein expression, sf9 insect cells were co-transfected with pIE1-Neo plasmid and pIEx-10 Ek/LIc-OX40L-Jagged-1 plasmid (at a ratio of 1:3). pIE1-Neo vector encoding antibiotic resistance gene G418 allowed for selection of stable transfectants. Thus, stable clones expressing truncated mouse OX40L-Jagged-1-Fc chimeric protein were selected with 300μg/m1 of G418 48 hours post transfection. In order to release protein from insect sells, the cells were incubated with Popculture reagent, buffered mixture of concentrated detergents formulated to extract proteins from insect cells directly in their culture medium. During 15 minute incubation, Insect PopCulture disrupts the cell membrane without denaturing proteins and protects them from the pH extremes in high-density culture media. To reduce viscosity, Benzonase Nuclease was added to the reagent. Benzonase degrades endogenous nucleic acids that may interfere with processing due to high viscosity and interaction with proteins of interest. Strep·Tactin® resin method was used for the protein purification. Purified truncated chimeric protein was resolved in 4-20% SDS-PAGE and comparison of expression/purification was done by Western blot using anti-StrepTag antibody (FIG.-12).

### Example 9

### Production and Expression of Human Truncated Chimeric Protein

A truncated human chimeric OX40L-Jagged-1-Fc construct was produced comprising the complete 133 amino acid ectodomain of human OX40L (coded by amino acids 51-183 of Uniprot ID: P23510) and a truncated Jagged-1 ectodomain (containing DSL domain and EGF like repeats 1-3 spanning 34-334 amino acids of P78504) linked by hinge region of human IgG1-Fc.

As described above, human Jagged-1 ectodomain is 1034 amino acids (34-1067aa) in length. Human Jagged-1 ectodomain consists of a DSL domain (amino acids 185-229) and 16 EGF-like repeats. Among these, DSL domain is indispensable for the interaction of Jagged-1 with Notch receptors. EGF-like repeats 1 and 2 help improve the affinity of the ligand-receptor interaction. However, the rest of the EGF-like repeats do not play a significant role in regulation of the binding of Jagged-1 with Notch receptors (Shimizu et al. Mouse jagged1 physically interacts with notch2 and other notch receptors. Assessment by quantitative methods. J Biol Chem. 1999 12; 274(46):32961-9).

### Cloning of Human Truncated OX40L-Jagged-1-Fc Chimeric Protein

PCR amplification of the truncated chimeric DNA fragment was accomplished using sense primer; 5′CCTTGATATCGATGTACAGGATGCAACTCCTGTCTTGCAT3′ (SEQ ID NO: 47) and anti-sense primer 5′GGCT CCATGGC TTCACAGTTGGGTCCTGAATAC3′(SEQ ID NO: 48). Underlined sequences indicate EcoRV and Nco1 sites respectively. Plasmid DNA of full length chimera was used as template for the PCR amplification. PCR condition was as follows: 1) Initial denaturation at 95° C. for 5 min, 2) Denaturation at 95° C. for 30s, 3) Annealing at 50° C. for 30s, 4) Extension at 72° C. for 30s for 35 cycles. PCR amplified chimera fragment ran on 1% agarose gel at 100V for 30 minutes was gel purified (FIG.-13). Chimera fragment and pFUSE-human IgG1-Fc2 vectors were digested with restriction enzyme EcoRV for 2 h at 37° C. Restricted DNA fragments were ran on 1% agarose gel at 100V for 30 minutes and gel purified. After purification, digested chimera fragment and pFUSE-human IgG1-Fc vectors were ligated with Quick ligase at a molar ration of 5:1 at room temperature for 30 min. Ligated pFUSE-human chimera was transformed in to DH5-α bacteria. Chimera clones were selected by ampicillin selection (100 μg/ml). PFUSE-Chimera plasmid was purified from E. coli. Orientation and reading frame of the chimera sequence was confirmed by Sanger DNA sequencing.

### Expression and Purification of Truncated Human Chimeric Protein in HEK293T Cells

Expression of full length and truncated hOX40L-Jagged-1-Fc chimeric proteins in HEK293T cells was accomplished as follows: 1×10⁶ HEK293T cells were transfected with 2μg of purified pFUSE-Chimera plasmid DNAs. Cells were cultured in DMEM-F12 media supplemented with 5% FBS. 72 h Post-transfection, Chimeric protein secreted from HEK293T cells were purified from culture supernatant using protein A beads by IgG affinity purification. Cell culture supernatants were incubated with protein-A agarose beads overnight at 4° C. Presence human IgG1 tag in chimeric protein will enable the binding of chimera with protein A. Beads were washed with 1X Phosphate Buffered Saline (PBS) to remove non-specific proteins. Chimeric protein was eluted by acidic elution buffer (pH 3.0) and immediately neutralized with basic neutralization buffer (pH 9.0). Purified chimeric protein was resolved in 4-20% SDS-PAGE and comparison of efficient secretion was done by Western blot using anti-human IgG1 antibody (FIG.-14). Comparative analysis showed more than 10 fold increased secretion efficiency of truncated chimera compared to full length chimera. Subsequently, a kill curve experiment was performed to determine the optimal antibiotic (Zeocin) concentration at which un-transfected HEK-293T cells will die after 10 days of selection. Based on this, stable chimera producing clones were selected by Zeocin selection (200 μg/ml) and screened by Flow cytometry and Western blot using human IgG1 specific antibody.

Human OX40L amino acid sequence Uniprot ID: P23510
(SEQ ID NO: 1)
MERVQPLEENVGNAARPRFERNKLLLVASVIQGLGLLLCFTYICLHFSALMVSHRYPRIQ
SIKVQFTEYKKEKGFILTSQKEDEIMKVQNNSVIINCDGFYLISLKGYFSQEVNISLHYQK
DEEPLFQLKKVRSVNSLMVASLTYKDKVYLNVTTDNTSLDDFHVNGGELILIHQNPGEF
CVL
Cytoplasmic domain-(1-23 amino acids)
Transmembrane domain-(25-50 amino acids)
Extracellular domain-(51-183 aa)
Human OX40L nucleotide coding sequence: NCBI Genbank ID:NM_003326
(SEQ ID NO: 2)
ATGGAAAGGGTCCAACCCCTGGAAGAGAATGTGGGAAATGCAGCCAGGCCAAGATT
CGAGAGGAACAAGCTATTGCTGGTGGCCTCTGTAATTCAGGGACTGGGGCTGCTCCT
GTGCTTCACCTACATCTGCCTGCACTTCTCTGCTCTTATGGTATCACATCGGTATCCT
CGAATTCAAAGTATCAAAGTACAATTTACCGAATATAAGAAGGAGAAAGGTTTCAT
CCTCACTTCCCAAAAGGAGGATGAAATCATGAAGGTGCAGAACAACTCAGTCATCA
TCAACTGTGATGGGTTTTATCTCATCTCCCTGAAGGGCTACTTCTCCCAGGAAGTCA
ACATTAGCCTTCATTACCAGAAGGATGAGGAGCCCCTCTTCCAACTGAAGAAGGTCA
GGTCTGTCAACTCCTTGATGGTGGCCTCTCTGACTTACAAAGACAAAGTCTACTTGA
ATGTGACCACTGACAATACCTCCCTGGATGACTTCCATGTGAATGGCGGAGAACTGA
TTCTTATCCATCAAAATCCTGGTGAATTCTGTGTCCTTTGA
Cytoplasmic domain-(1-69 bases)
Transmembrane domain-(70-150 bases)
Extracellular domain-(151-552 bases)
Human Jagged-1 amino acid sequence Uniprot ID: P78504
(SEQ ID NO: 5)
MRSPRTRGRSGRPLSLLLALLCALRAKVCGASGQFELEILSMQNVNGELQNGNCCGGA
RNPGDRKCTRDECDTYFKVCLKEYQSRVTAGGPCSFGSGSTPVIGGNTFNLKASRGNDR
NRIVLPFSFAWPRSYTLLVEAWDSSNDTVQPDSIIEKASHSGMINPSRQWQTLKQNTGV
AHFEYQIRVTCDDYYYGFGCNKFCRPRDDFFGHYACDQNGNKTCMEGWMGPECNRAI
CRQGCSPKHGSCKLPGDCRCQYGWQGLYCDKCIPHPGCVHGICNEPWQCLCETNWGG
QLCDKDLNYCGTHQPCLNGGTCSNTGPDKYQCSCPEGYSGPNCEIAEHACLSDPCHNR
GSCKETSLGFECECSPGWTGPTCSTNIDDCSPNNCSHGGTCQDLVNGFKCVCPPQWTGK
TCQLDANECEAKPCVNAKSCKNLIASYYCDCLPGWMGQNCDININDCLGQCQNDASCR
DLVNGYRCICPPGYAGDHCERDIDECASNPCLNGGHCQNEINRFQCLCPTGFSGNLCQL
DIDYCEPNPCQNGAQCYNRASDYFCKCPEDYEGKNCSHLKDHCRTTPCEVIDSCTVAM
ASNDTPEGVRYISSNVCGPHGKCKSQSGGKFTCDCNKGFTGTYCHENINDCESNPCRNG
GTCIDGVNSYKCICSDGWEGAYCETNINDCSQNPCHNGGTCRDLVNDFYCDCKNGWK
GKTCHSRDSQCDEATCNNGGTCYDEGDAFKCMCPGGWEGTTCNIARNSSCLPNPCHNG
GTCVVNGESFTCVCKEGWEGPICAQNTNDCSPHPCYNSGTCVDGDNWYRCECAPGFA
GPDCRININECQSSPCAFGATCVDEINGYRCVCPPGHSGAKCQEVSGRPCITMGSVIPDG
AKWDDDCNTCQCLNGRIACSKVWCGPRPCLLHKGHSECPSGQSCIPILDDQCFVHPCTG
VGECRSSSLQPVKTKCTSDSYYQDNCANITFTFNKEMMSPGLTTEHICSELRNLNILKNV
SAEYSIYIACEPSPSANNEIHVAISAEDIRDDGNPIKEITDKIIDLVSKRDGNSSLIAAVAEV
RVQRRPLKNRTDFLVPLLSSVLTVAWICCLVTAFYWCLRKRRKPGSHTHSASEDNTTNN
VREQLNQIKNPIEKHGANTVPIKDYENKNSKMSKIRTHNSEVEEDDMDKHQQKARFAK
QPAYTLVDREEKPPNGTPTKHPNWTNKQDNRDLESAQSLNRMEYIV
Signal peptide-(1-32 amino acids)
Extracellular domain-(33-1067 amino acids)
Transmembrane domain-(1068-1093 amino acids)
Cytoplasmic domain-(1094-1218 amino acids)
Human Jagged-1 nucleotide coding sequence NCBI Genbank ID: NM_000214
(SEQ ID NO: 6)
ATGCGTTCCCCACGGACGCGCGGCCGGTCCGGGCGCCCCCTAAGCCTCCTGCTCGCC
CTGCTCTGTGCCCTGCGAGCCAAGGTGTGTGGGGCCTCGGGTCAGTTCGAGTTGGAG
ATCCTGTCCATGCAGAACGTGAACGGGGAGCTGCAGAACGGGAACTGCTGCGGCGG
CGCCCGGAACCCGGGAGACCGCAAGTGCACCCGCGACGAGTGTGACACATACTTCA
AAGTGTGCCTCAAGGAGTATCAGTCCCGCGTCACGGCCGGGGGGCCCTGCAGCTTC
GGCTCAGGGTCCACGCCTGTCATCGGGGGCAACACCTTCAACCTCAAGGCCAGCCG
CGGCAACGACCGCAACCGCATCGTGCTGCCTTTCAGTTTCGCCTGGCCGAGGTCCTA
TACGTTGCTTGTGGAGGCGTGGGATTCCAGTAATGACACCGTTCAACCTGACAGTAT
TATTGAAAAGGCTTCTCACTCGGGCATGATCAACCCCAGCCGGCAGTGGCAGACGC
TGAAGCAGAACACGGGCGTTGCCCACTTTGAGTATCAGATCCGCGTGACCTGTGATG
ACTACTACTATGGCTTTGGCTGCAATAAGTTCTGCCGCCCCAGAGATGACTTCTTTG
GACACTATGCCTGTGACCAGAATGGCAACAAAACTTGCATGGAAGGCTGGATGGGC
CCCGAATGTAACAGAGCTATTTGCCGACAAGGCTGCAGTCCTAAGCATGGGTCTTGC
AAACTCCCAGGTGACTGCAGGTGCCAGTACGGCTGGCAAGGCCTGTACTGTGATAA
GTGCATCCCACACCCGGGATGCGTCCACGGCATCTGTAATGAGCCCTGGCAGTGCCT
CTGTGAGACCAACTGGGGCGGCCAGCTCTGTGACAAAGATCTCAATTACTGTGGGA
CTCATCAGCCGTGTCTCAACGGGGGAACTTGTAGCAACACAGGCCCTGACAAATAT
CAGTGTTCCTGCCCTGAGGGGTATTCAGGACCCAACTGTGAAATTGCTGAGCACGCC
TGCCTCTCTGATCCCTGTCACAACAGAGGCAGCTGTAAGGAGACCTCCCTGGGCTTT
GAGTGTGAGTGTTCCCCAGGCTGGACCGGCCCCACATGCTCTACAAACATTGATGAC
TGTTCTCCTAATAACTGTTCCCACGGGGGCACCTGCCAGGACCTGGTTAACGGATTT
AAGTGTGTGTGCCCCCCACAGTGGACTGGGAAAACGTGCCAGTTAGATGCAAATGA
ATGTGAGGCCAAACCTTGTGTAAACGCCAAATCCTGTAAGAATCTCATTGCCAGCTA
CTACTGCGACTGTCTTCCCGGCTGGATGGGTCAGAATTGTGACATAAATATTAATGA
CTGCCTTGGCCAGTGTCAGAATGACGCCTCCTGTCGGGATTTGGTTAATGGTTATCG
CTGTATCTGTCCACCTGGCTATGCAGGCGATCACTGTGAGAGAGACATCGATGAATG
TGCCAGCAACCCCTGTTTGAATGGGGGTCACTGTCAGAATGAAATCAACAGATTCCA
GTGTCTGTGTCCCACTGGTTTCTCTGGAAACCTCTGTCAGCTGGACATCGATTATTGT
GAGCCTAATCCCTGCCAGAACGGTGCCCAGTGCTACAACCGTGCCAGTGACTATTTC
TGCAAGTGCCCCGAGGACTATGAGGGCAAGAACTGCTCACACCTGAAAGACCACTG
CCGCACGACCCCCTGTGAAGTGATTGACAGCTGCACAGTGGCCATGGCTTCCAACG
ACACACCTGAAGGGGTGCGGTATATTTCCTCCAACGTCTGTGGTCCTCACGGGAAGT
GCAAGAGTCAGTCGGGAGGCAAATTCACCTGTGACTGTAACAAAGGCTTCACGGGA
ACATACTGCCATGAAAATATTAATGACTGTGAGAGCAACCCTTGTAGAAACGGTGG
CACTTGCATCGATGGTGTCAACTCCTACAAGTGCATCTGTAGTGACGGCTGGGAGGG
GGCCTACTGTGAAACCAATATTAATGACTGCAGCCAGAACCCCTGCCACAATGGGG
GCACGTGTCGCGACCTGGTCAATGACTTCTACTGTGACTGTAAAAATGGGTGGAAAG
GAAAGACCTGCCACTCACGTGACAGTCAGTGTGATGAGGCCACGTGCAACAACGGT
GGCACCTGCTATGATGAGGGGGATGCTTTTAAGTGCATGTGTCCTGGCGGCTGGGAA
GGAACAACCTGTAACATAGCCCGAAACAGTAGCTGCCTGCCCAACCCCTGCCATAA
TGGGGGCACATGTGTGGTCAACGGCGAGTCCTTTACGTGCGTCTGCAAGGAAGGCT
GGGAGGGGCCCATCTGTGCTCAGAATACCAATGACTGCAGCCCTCATCCCTGTTACA
ACAGCGGCACCTGTGTGGATGGAGACAACTGGTACCGGTGCGAATGTGCCCCGGGT
TTTGCTGGGCCCGACTGCAGAATAAACATCAATGAATGCCAGTCTTCACCTTGTGCC
TTTGGAGCGACCTGTGTGGATGAGATCAATGGCTACCGGTGTGTCTGCCCTCCAGGG
CACAGTGGTGCCAAGTGCCAGGAAGTTTCAGGGAGACCTTGCATCACCATGGGGAG
TGTGATACCAGATGGGGCCAAATGGGATGATGACTGTAATACCTGCCAGTGCCTGA
ATGGACGGATCGCCTGCTCAAAGGTCTGGTGTGGCCCTCGACCTTGCCTGCTCCACA
AAGGGCACAGCGAGTGCCCCAGCGGGCAGAGCTGCATCCCCATCCTGGACGACCAG
TGCTTCGTCCACCCCTGCACTGGTGTGGGCGAGTGTCGGTCTTCCAGTCTCCAGCCG
GTGAAGACAAAGTGCACCTCTGACTCCTATTACCAGGATAACTGTGCGAACATCACA
TTTACCTTTAACAAGGAGATGATGTCACCAGGTCTTACTACGGAGCACATTTGCAGT
GAATTGAGGAATTTGAATATTTTGAAGAATGTTTCCGCTGAATATTCAATCTACATC
GCTTGCGAGCCTTCCCCTTCAGCGAACAATGAAATACATGTGGCCATTTCTGCTGAA
GATATACGGGATGATGGGAACCCGATCAAGGAAATCACTGACAAAATAATCGATCT
TGTTAGTAAACGTGATGGAAACAGCTCGCTGATTGCTGCCGTTGCAGAAGTAAGAGT
TCAGAGGCGGCCTCTGAAGAACAGAACAGATTTCCTTGTTCCCTTGCTGAGCTCTGT
CTTAACTGTGGCTTGGATCTGTTGCTTGGTGACGGCCTTCTACTGGTGCCTGCGGAA
GCGGCGGAAGCCGGGCAGCCACACACACTCAGCCTCTGAGGACAACACCACCAACA
ACGTGCGGGAGCAGCTGAACCAGATCAAAAACCCCATTGAGAAACATGGGGCCAAC
ACGGTCCCCATCAAGGATTATGAGAACAAGAACTCCAAAATGTCTAAAATAAGGAC
ACACAATTCTGAAGTAGAAGAGGACGACATGGACAAACACCAGCAGAAAGCCCGG
TTTGCCAAGCAGCCGGCGTACACGCTGGTAGACAGAGAAGAGAAGCCCCCCAACGG
CACGCCGACAAAACACCCAAACTGGACAAACAAACAGGACAACAGAGACTTGGAA
AGTGCCCAGAGCTTAAACCGAATGGAGTACATCGTATGA
Signal peptide-(1-99 bases)
Extracellular domain-(100-3201 bases)
Transmembrane domain-(3202-3279 bases)
Cytoplasmic domain-(3280- 3657 bases)
Mouse OX40L amino acid sequence Uniprot ID: P43488
(SEQ ID NO: 3)
MEGEGVQPLDENLENGSRPRFKWKKTLRLVVSGIKGAGMLLCFIYVCLQLSSSPAKDPP
IQRLRGAVTRCEDGQLFISSYKNEYQTMEVQNNSVVIKCDGLYIIYLKGSFFQEVKIDLH
FREDHNPISIPMLNDGRRIVFTVVASLAFKDKVYLTVNAPDTLCEHLQINDGELIVVQLTP
GYCAPEGSYHSTVNQVPL
Cytoplasmic domain-(1-28 amino acids)
Transmembrane domain-(29-50 amino acids)
Extracellular domain-(51-198 amino acids)
Mouse OX40L nucleotide sequence NCBI Genbank ID: NM_009452
(SEQ ID NO: 4)
ATGGAAGGGGAAGGGGTTCAACCCCTGGATGAGAATCTGGAAAACGGATCAAGGCC
AAGATTCAAGTGGAAGAAGACGCTAAGGCTGGTGGTCTCTGGGATCAAGGGAGCAG
GGATGCTTCTGTGCTTCATCTATGTCTGCCTGCAACTCTCTTCCTCTCCGGCAAAGGA
CCCTCCAATCCAAAGACTCAGAGGAGCAGTTACCAGATGTGAGGATGGGCAACTAT
TCATCAGCTCATACAAGAATGAGTATCAAACTATGGAGGTGCAGAACAATTCGGTT
GTCATCAAGTGCGATGGGCTTTATATCATCTACCTGAAGGGCTCCTTTTTCCAGGAG
GTCAAGATTGACCTTCATTTCCGGGAGGATCATAATCCCATCTCTATTCCAATGCTG
AACGATGGTCGAAGGATTGTCTTCACTGTGGTGGCCTCTTTGGCTTTCAAAGATAAA
GTTTACCTGACTGTAAATGCTCCTGATACTCTCTGCGAACACCTCCAGATAAATGAT
GGGGAGCTGATTGTTGTCCAGCTAACGCCTGGATACTGTGCTCCTGAAGGATCTTAC
CACAGCACTGTGAACCAAGTACCACTGTGA
Cytoplasmic domain-(1-84 bases)
Transmembrane domain-(85-150 bases)
Extracellular domain-(151-597 bases)
Mouse Jagged1 amino acid sequence Uniprot ID: Q9QXX0
(SEQ ID NO: 7)
MRSPRTRGRPGRPLSLLLALLCALRAKVCGASGQFELEILSMQNVNGELQNGNCCGGV
RNPGDRKCTRDECDTYFKVCLKEYQSRVTAGGPCSFGSGSTPVIGGNTFNLKASRGNDR
NRIVLPFSFAWPRSYTLLVEAWDSSNDTIQPDSIIEKASHSGMINPSRQWQTLKQNTGIAH
FEYQIRVTCDDHYYGFGCNKFCRPRDDFFGHYACDQNGNKTCMEGWMGPDCNKAICR
QGCSPKHGSCKLPGDCRCQYGWQGLYCDKCIPHPGCVHGTCNEPWQCLCETNWGGQL
CDKDLNYCGTHQPCLNRGTCSNTGPDKYQCSCPEGYSGPNCEIAEHACLSDPCHNRGSC
KETSSGFECECSPGWTGPTCSTNIDDCSPNNCSHGGTCQDLVNGFKCVCPPQWTGKTCQ
LDANECEAKPCVNARSCKNLIASYYCDCLPGWMGQNCDININDCLGQCQNDASCRDLV
NGYRCICPPGYAGDHCERDIDECASNPCLNGGHCQNEINRFQCLCPTGFSGNLCQLDIDY
CEPNPCQNGAQCYNRASDYFCKCPEDYEGKNCSHLKDHCRTTTCEVIDSCTVAMASND
TPEGVRYISSNVCGPHGKCKSQSGGKFTCDCNKGFTGTYCHENINDCESNPCKNGGTCI
DGVNSYKCICSDGWEGAHCENNINDCSQNPCHYGGTCRDLVNDFYCDCKNGWKGKTC
HSRDSQCDEATCNNGGTCYDEVDTFKCMCPGGWEGTTCNIARNSSCLPNPCHNGGTCV
VNGDSFTCVCKEGWEGPICTQNTNDCSPHPCYNSGTCVDGDNWYRCECAPGFAGPDCR
ININECQSSPCAFGATCVDEINGYQCICPPGHSGAKCHEVSGRSCITMGRVILDGAKWDD
DCNTCQCLNGRVACKVWCGPRPCRLHKSHNECPSGQSCIPVLDDQCFVRPCTGVGEC
RSSSLQPVKTKCTSDSYYQDNCANITFTFNKEMMSPGLTTEHICSELRNLNILKNVSAEY
SIYIACEPSLSANNEIHVAISAEDIRDDGNPVKEITDKIIDLVSKRDGNSSLIAAVAEVRVQ
RRPLKNRTDFLVPLLSSVLTVAWVCCLVTAFYWCVRKRRKPSSHTHSAPEDNTTNNVR
EQLNQIKNPIEKHGANTVPIKDYENKNSKMSKIRTHNSEVEEDDMDKHQQKVRFAKQP
VYTLVDREEKAPSGTPTKHPNWTNKQDNRDLESAQSLNRMEYIV
Signal peptide-(1-32 amino acids)
Extracellular domain-(33-1067 amino acids)
Transmembrane domain-(1068-1093 amino acids)
Cytoplasmic domain-(1094-1218 amino acids)
Mouse Jagged1 nucleotide sequence NCBI Genbank ID: NM_013822
(SEQ ID NO: 8)
ATGCGGTCCCCACGGACGCGCGGCCGGCCCGGGCGCCCCCTGAGTCTTCTGCTCGCC
CTGCTCTGTGCCCTGCGAGCCAAGGTGTGCGGGGCCTCGGGTCAGTTTGAGCTGGAG
ATCCTGTCCATGCAGAACGTGAATGGAGAGCTACAGAATGGGAACTGTTGTGGTGG
AGTCCGGAACCCTGGCGACCGCAAGTGCACCCGCGACGAGTGTGATACGTACTTCA
AAGTGTGCCTCAAGGAGTATCAGTCCCGCGTCACTGCCGGGGGACCCTGCAGCTTCG
GCTCAGGGTCTACGCCTGTCATCGGGGGTAACACCTTCAATCTCAAGGCCAGCCGTG
GCAACGACCGTAATCGCATCGTACTGCCTTTCAGTTTCGCCTGGCCGAGGTCCTACA
CTTTGCTGGTGGAGGCCTGGGATTCCAGTAATGACACTATTCAACCTGATAGCATAA
TTGAAAAGGCTTCTCACTCAGGCATGATAAACCCTAGCCGGCAATGGCAGACACTG
AAACAAAACACAGGGATTGCCCACTTCGAGTATCAGATCCGAGTGACCTGTGATGA
CCACTACTATGGCTTTGGCTGCAATAAGTTCTGTCGTCCCAGAGATGACTTCTTTGGA
CATTATGCCTGTGACCAGAACGGCAACAAAACTTGCATGGAAGGCTGGATGGGTCC
TGATTGCAACAAAGCTATCTGCCGACAGGGCTGCAGTCCCAAGCATGGGTCTTGTAA
ACTTCCAGGTGACTGCAGGTGCCAGTACGGTTGGCAGGGCCTGTACTGCGACAAGT
GCATCCCGCACCCAGGATGTGTCCACGGCACCTGCAATGAACCCTGGCAGTGCCTCT
GTGAGACCAACTGGGGTGGACAGCTCTGTGACAAAGATCTGAATTACTGTGGGACT
CATCAGCCCTGTCTCAACCGGGGAACATGTAGCAACACTGGGCCTGACAAATACCA
GTGCTCCTGCCCAGAGGGCTACTCGGGCCCCAACTGTGAAATTGCTGAGCATGCTTG
TCTCTCTGACCCCTGCCATAACCGAGGCAGCTGCAAGGAGACCTCCTCAGGCTTTGA
GTGTGAGTGTTCTCCAGGCTGGACTGGCCCCACGTGTTCCACAAACATCGATGACTG
TTCTCCAAATAACTGTTCCCATGGGGGCACCTGCCAGGATCTGGTGAATGGATTCAA
GTGTGTGTGCCCGCCCCAGTGGACTGGCAAGACTTGTCAGTTAGATGCAAATGAGTG
CGAGGCCAAACCTTGTGTAAATGCCAGATCCTGTAAGAATCTGATTGCCAGCTACTA
CTGTGATTGCCTTCCTGGCTGGATGGGTCAGAACTGTGACATAAATATCAATGACTG
CCTTGGCCAGTGTCAGAATGACGCCTCCTGTCGGGATTTGGTTAATGGTTATCGCTG
TATCTGTCCACCTGGCTATGCAGGCGATCACTGTGAGAGAGACATCGATGAGTGTGC
TAGCAACCCCTGCTTGAATGGGGGTCACTGTCAGAATGAAATCAACAGATTCCAGTG
TCTCTGTCCCACTGGTTTCTCTGGAAACCTCTGTCAGCTGGACATCGATTACTGCGAG
CCCAACCCTTGCCAGAATGGCGCCCAGTGCTACAATCGTGCCAGTGACTATTTCTGC
AAGTGCCCCGAGGACTATGAGGGCAAGAACTGCTCACACCTGAAAGACCACTGCCG
TACCACCACCTGCGAAGTGATTGACAGCTGCACTGTGGCCATGGCCTCCAACGACAC
GCCTGAAGGGGTGCGGTATATCTCTTCTAACGTCTGTGGTCCCCATGGGAAGTGCAA
GAGCCAGTCGGGAGGCAAATTCACCTGTGACTGTAACAAAGGCTTCACCGGCACCT
ACTGCCATGAAAATATCAACGACTGCGAGAGCAACCCCTGTAAAAACGGTGGCACC
TGCATCGATGGCGTTAACTCCTACAAGTGTATCTGTAGTGACGGCTGGGAGGGAGCG
CACTGTGAGAACAACATAAATGACTGTAGCCAGAACCCTTGTCACTACGGGGGTAC
ATGTCGAGACCTGGTCAATGACTTTTACTGTGACTGCAAAAATGGCTGGAAAGGAA
AGACTTGCCATTCCCGTGACAGCCAGTGTGACGAAGCCACGTGTAATAATGGTGGTA
CCTGCTATGATGAAGTGGACACGTTTAAGTGCATGTGTCCCGGTGGCTGGGAAGGA
ACAACCTGTAATATAGCTAGAAACAGTAGCTGCCTGCCGAACCCCTGTCATAATGGA
GGTACCTGCGTGGTCAATGGAGACTCCTTCACCTGTGTCTGCAAAGAAGGCTGGGAG
GGGCCTATTTGTACTCAAAATACCAACGACTGCAGTCCCCATCCTTGTTACAATAGC
GGGACCTGTGTGGACGGAGACAACTGGTATCGGTGCGAATGTGCCCCGGGTTTTGCT
GGGCCAGACTGCAGGATAAACATCAATGAGTGCCAGTCTTCCCCTTGTGCCTTTGGG
GCCACCTGTGTGGATGAGATCAATGGCTACCAGTGTATCTGCCCTCCAGGACATAGT
GGTGCCAAGTGCCATGAAGTTTCAGGGCGATCTTGCATCACCATGGGGAGAGTGAT
ACTTGATGGGGCCAAGTGGGATGATGACTGTAACACCTGCCAGTGCCTGAATGGAC
GGGTGGCCTGCTCCAAGGTCTGGTGTGGCCCGAGACCTTGCAGGCTCCACAAAAGC
CACAATGAGTGCCCCAGTGGGCAGAGCTGCATCCCGGTCCTGGATGACCAGTGTTTC
GTGCGCCCCTGCACTGGTGTTGGCGAGTGTCGGTCCTCCAGCCTCCAGCCAGTGAAG
ACCAAGTGCACATCTGACTCCTATTACCAGGATAACTGTGCAAACATCACTTTCACC
TTTAACAAAGAGATGATGTCTCCAGGTCTTACCACCGAACACATTTGCAGCGAATTG
AGGAATTTGAATATCCTGAAGAATGTTTCTGCTGAATATTCGATCTACATAGCCTGT
GAGCCTTCCCTGTCAGCAAACAATGAAATACACGTGGCCATCTCTGCAGAAGACAT
CCGGGATGATGGGAACCCTGTCAAGGAAATTACCGATAAAATAATAGATCTCGTTA
GTAAACGGGATGGAAACAGCTCACTTATTGCTGCGGTTGCAGAAGTCAGAGTTCAG
AGGCGTCCTCTGAAAAACAGAACAGATTTCCTGGTTCCTCTGCTGAGCTCTGTCTTA
ACAGTGGCTTGGGTCTGTTGCTTGGTGACAGCCTTCTACTGGTGTGTAAGGAAGCGG
CGGAAGCCCAGCAGCCACACTCACTCCGCCCCCGAGGACAACACCACCAACAATGT
GCGGGAGCAGCTGAACCAAATCAAAAACCCCATCGAGAAACACGGAGCCAACACG
GTCCCCATTAAGGATTACGAGAACAAAAACTCGAAAATGTCAAAAATCAGGACACA
CAACTCGGAAGTGGAGGAGGATGACATGGATAAACACCAGCAGAAAGTCCGCTTTG
CCAAACAGCCAGTGTATACGCTGGTAGACAGAGAGGAGAAGGCCCCCAGCGGCAC
GCCGACAAAACACCCGAACTGGACAAATAAACAGGACAACAGAGACTTGGAAAGT
GCCCAGAGCTTGAACCGGATGGAATACATCGTATAG
Signal peptide-(1-96 bases)
Extracellular domain-(97-3198 bases)
Transmembrane domain-(3199-3276 bases)
Cytoplasmic domain-(3277- 3657 bases)
Human OX40L-JAG1-Fc Chimera nucleotide sequence
(SEQ ID NO: 10)
ATGTACAGGATGCAACTCCTGTCTTGCATTGCACTAAGTCTTGCACTTGTCACGAATT
CGATGGTATCACATCGGTATCCTCGAATTCAAAGTATCAAAGTACAATTTACCGAAT
ATAAGAAGGAGAAAGGTTTCATCCTCACTTCCCAAAAGGAGGATGAAATCATGAAG
GTGCAGAACAACTCAGTCATCATCAACTGTGATGGGTTTTATCTCATCTCCCTGAAG
GGCTACTTCTCCCAGGAAGTCAACATTAGCCTTCATTACCAGAAGGATGAGGAGCCC
CTCTTCCAACTGAAGAAGGTCAGGTCTGTCAACTCCTTGATGGTGGCCTCTCTGACTT
ACAAAGACAAAGTCTACTTGAATGTGACCACTGACAATACCTCCCTGGATGACTTCC
ATGTGAATGGCGGAGAACTGATTCTTATCCATCAAAATCCTGGTGAATTCTGTGTCC
TTTGGGCACGGTGGGCATGTGTGAGTTTTGTCCAGTTCGAGTTGGAGATCCTGTCCA
TGCAGAACGTGAACGGGGAGCTGCAGAACGGGAACTGCTGCGGCGGCGCCCGGAA
CCCGGGAGACCGCAAGTGCACCCGCGACGAGTGTGACACATACTTCAAAGTGTGCC
TCAAGGAGTATCAGTCCCGCGTCACGGCCGGGGGGCCCTGCAGCTTCGGCTCAGGG
TCCACGCCTGTCATCGGGGGCAACACCTTCAACCTCAAGGCCAGCCGCGGCAACGA
CCGCAACCGCATCGTGCTGCCTTTCAGTTTCGCCTGGCCGAGGTCCTATACGTTGCTT
GTGGAGGCGTGGGATTCCAGTAATGACACCGTTCAACCTGACAGTATTATTGAAAA
GGCTTCTCACTCGGGCATGATCAACCCCAGCCGGCAGTGGCAGACGCTGAAGCAGA
ACACGGGCGTTGCCCACTTTGAGTATCAGATCCGCGTGACCTGTGATGACTACTACT
ATGGCTTTGGCTGCAATAAGTTCTGCCGCCCCAGAGATGACTTCTTTGGACACTATG
CCTGTGACCAGAATGGCAACAAAACTTGCATGGAAGGCTGGATGGGCCCCGAATGT
AACAGAGCTATTTGCCGACAAGGCTGCAGTCCTAAGCATGGGTCTTGCAAACTCCCA
GGTGACTGCAGGTGCCAGTACGGCTGGCAAGGCCTGTACTGTGATAAGTGCATCCC
ACACCCGGGATGCGTCCACGGCATCTGTAATGAGCCCTGGCAGTGCCTCTGTGAGAC
CAACTGGGGCGGCCAGCTCTGTGACAAAGATCTCAATTACTGTGGGACTCATCAGCC
GTGTCTCAACGGGGGAACTTGTAGCAACACAGGCCCTGACAAATATCAGTGTTCCTG
CCCTGAGGGGTATTCAGGACCCAACTGTGAAATTGCTGAGCACGCCTGCCTCTCTGA
TCCCTGTCACAACAGAGGCAGCTGTAAGGAGACCTCCCTGGGCTTTGAGTGTGAGTG
TTCCCCAGGCTGGACCGGCCCCACATGCTCTACAAACATTGATGACTGTTCTCCTAA
TAACTGTTCCCACGGGGGCACCTGCCAGGACCTGGTTAACGGATTTAAGTGTGTGTG
CCCCCCACAGTGGACTGGGAAAACGTGCCAGTTAGATGCAAATGAATGTGAGGCCA
AACCTTGTGTAAACGCCAAATCCTGTAAGAATCTCATTGCCAGCTACTACTGCGACT
GTCTTCCCGGCTGGATGGGTCAGAATTGTGACATAAATATTAATGACTGCCTTGGCC
AGTGTCAGAATGACGCCTCCTGTCGGGATTTGGTTAATGGTTATCGCTGTATCTGTCC
ACCTGGCTATGCAGGCGATCACTGTGAGAGAGACATCGATGAATGTGCCAGCAACC
CCTGTTTGAATGGGGGTCACTGTCAGAATGAAATCAACAGATTCCAGTGTCTGTGTC
CCACTGGTTTCTCTGGAAACCTCTGTCAGCTGGACATCGATTATTGTGAGCCTAATCC
CTGCCAGAACGGTGCCCAGTGCTACAACCGTGCCAGTGACTATTTCTGCAAGTGCCC
CGAGGACTATGAGGGCAAGAACTGCTCACACCTGAAAGACCACTGCCGCACGACCC
CCTGTGAAGTGATTGACAGCTGCACAGTGGCCATGGCTTCCAACGACACACCTGAA
GGGGTGCGGTATATTTCCTCCAACGTCTGTGGTCCTCACGGGAAGTGCAAGAGTCAG
TCGGGAGGCAAATTCACCTGTGACTGTAACAAAGGCTTCACGGGAACATACTGCCA
TGAAAATATTAATGACTGTGAGAGCAACCCTTGTAGAAACGGTGGCACTTGCATCG
ATGGTGTCAACTCCTACAAGTGCATCTGTAGTGACGGCTGGGAGGGGGCCTACTGTG
AAACCAATATTAATGACTGCAGCCAGAACCCCTGCCACAATGGGGGCACGTGTCGC
GACCTGGTCAATGACTTCTACTGTGACTGTAAAAATGGGTGGAAAGGAAAGACCTG
CCACTCACGTGACAGTCAGTGTGATGAGGCCACGTGCAACAACGGTGGCACCTGCT
ATGATGAGGGGGATGCTTTTAAGTGCATGTGTCCTGGCGGCTGGGAAGGAACAACC
TGTAACATAGCCCGAAACAGTAGCTGCCTGCCCAACCCCTGCCATAATGGGGGCAC
ATGTGTGGTCAACGGCGAGTCCTTTACGTGCGTCTGCAAGGAAGGCTGGGAGGGGC
CCATCTGTGCTCAGAATACCAATGACTGCAGCCCTCATCCCTGTTACAACAGCGGCA
CCTGTGTGGATGGAGACAACTGGTACCGGTGCGAATGTGCCCCGGGTTTTGCTGGGC
CCGACTGCAGAATAAACATCAATGAATGCCAGTCTTCACCTTGTGCCTTTGGAGCGA
CCTGTGTGGATGAGATCAATGGCTACCGGTGTGTCTGCCCTCCAGGGCACAGTGGTG
CCAAGTGCCAGGAAGTTTCAGGGAGACCTTGCATCACCATGGGGAGTGTGATACCA
GATGGGGCCAAATGGGATGATGACTGTAATACCTGCCAGTGCCTGAATGGACGGAT
CGCCTGCTCAAAGGTCTGGTGTGGCCCTCGACCTTGCCTGCTCCACAAAGGGCACAG
CGAGTGCCCCAGCGGGCAGAGCTGCATCCCCATCCTGGACGACCAGTGCTTCGTCCA
CCCCTGCACTGGTGTGGGCGAGTGTCGGTCTTCCAGTCTCCAGCCGGTGAAGACAAA
GTGCACCTCTGACTCCTATTACCAGGATAACTGTGCGAACATCACATTTACCTTTAA
CAAGGAGATGATGTCACCAGGTCTTACTACGGAGCACATTTGCAGTGAATTGAGGA
ATTTGAATATTTTGAAGAATGTTTCCGCTGAATATTCAATCTACATCGCTTGCGAGCC
TTCCCCTTCAGCGAACAATGAAATACATGTGGCCATTTCTGCTGAAGATATACGGGA
TGATGGGAACCCGATCAAGGAAATCACTGACAAAATAATCGATCTTGTTAGTAAAC
GTGATGGAAACAGCTCGCTGATTGCTGCCGTTGCAGAAGTAAGAGTTCAGAGGCGG
CCTCTGAAGAACAGAACAGATGACAAAACTCACACATGCCCACCGTGCCCAGCACC
TGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGT
CAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGGCAGCAG
GGGAACGTCTTCTCATGCTCCGTGATGCACGAGGCTCTGCACAACCACTACACGCAG
AAGAGCCTCTCCCTGTCTCCGGGTAAATGA
IL-2 signal sequence-(1-60 bases)
OX40L-extracellular domain-(61-459 bases)
Fc-Linker-(460-489 bases)
Jagged1 extracellular domain-(490-3591 bases)
Human IgG1-Fc2-(3592-4275 bases)
Human OX40L-JAG1-Fc Chimera amino acid sequence
(SEQ ID NO: 9)
MVSHRYPRIQSIKVQFTEYKKEKGFILTSQKEDEIMKVQNNSVIINCDGFYLISLKGYFSQ
EVNISLHYQKDEEPLFQLKKVRSVNSLMVASLTYKDKVYLNVTTDNTSLDDFHVNGGE
LILIHQNPGEFCVLDKTHTCPPCPQFELEILSMQNVNGELQNGNCCGGARNPGDRKCTR
DECDTYFKVCLKEYQSRVTAGGPCSFGSGSTPVIGGNTFNLKASRGNDRNRIVLPFSFA
WPRSYTLLVEAWDSSNDTVQPDSIIEKASHSGMINPSRQWQTLKQNTGVAHFEYQIRVT
CDDYYYGFGCNKFCRPRDDFFGHYACDQNGNKTCMEGWMGPECNRAICRQGCSPKH
GSCKLPGDCRCQYGWQGLYCDKCIPHPGCVHGICNEPWQCLCETNWGGQLCDKDLNY
CGTHQPCLNGGTCSNTGPDKYQCSCPEGYSGPNCEIAEHACLSDPCHNRGSCKETSLGF
ECECSPGWTGPTCSTNIDDCSPNNCSHGGTCQDLVNGFKCVCPPQWTGKTCQLDANEC
EAKPCVNAKSCKNLIASYYCDCLPGWMGQNCDININDCLGQCQNDASCRDLVNGYRCI
CPPGYAGDHCERDIDECASNPCLNGGHCQNEINRFQCLCPTGFSGNLCQLDIDYCEPNPC
QNGAQCYNRASDYFCKCPEDYEGKNCSHLKDHCRTTPCEVIDSCTVAMASNDTPEGVR
YISSNVCGPHGKCKSQSGGKFTCDCNKGFTGTYCHENINDCESNPCRNGGTCIDGVNSY
KCICSDGWEGAYCETNINDCSQNPCHNGGTCRDLVNDFYCDCKNGWKGKTCHSRDSQ
CDEATCNNGGTCYDEGDAFKCMCPGGWEGTTCNIARNSSCLPNPCHNGGTCVVNGESF
TCVCKEGWEGPICAQNTNDCSPHPCYNSGTCVDGDNWYRCECAPGFAGPDCRININEC
QSSPCAFGATCVDEINGYRCVCPPGHSGAKCQEVSGRPCITMGSVIPDGAKWDDDCNTC
QCLNGRIACSKVWCGPRPCLLHKGHSECPSGQSCIPILDDQCFVHPCTGVGECRSSSLQP
VKTKCTSDSYYQDNCANITFTFNKEMMSPGLTTEHICSELRNLNILKNVSAEYSIYIACEP
SPSANNEIHVAISAEDIRDDGNPIKEITDKIIDLVSKRDGNSSLIAAVAEVRVQRRPLKNRT
DDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
OX40L extracellular domain-(1-133 amino acids)
Fc linker-(134-144 amino acids)
Jagged1 extracellular domain-(145-1177 amino acids)
Human IgG1-Fc2-(1178-1404 amino acids)
Mouse OX40L-JAG1-Fc Chimera nucleotide sequence
(SEQ ID NO: 12)
ATGTACAGGATGCAACTCCTGTCTTGCATTGCACTAAGTCTTGCACTTGTCACGAATT
CGCAACTCTCTTCCTCTCCGGCAAAGGACCCTCCAATCCAAAGACTCAGAGGAGCA
GTTACCAGATGTGAGGATGGGCAACTATTCATCAGCTCATACAAGAATGAGTATCA
AACTATGGAGGTGCAGAACAATTCGGTTGTCATCAAGTGCGATGGGCTTTATATCAT
CTACCTGAAGGGCTCCTTTTTCCAGGAGGTCAAGATTGACCTTCATTTCCGGGAGGA
TCATAATCCCATCTCTATTCCAATGCTGAACGATGGTCGAAGGATTGTCTTCACTGTG
GTGGCCTCTTTGGCTTTCAAAGATAAAGTTTACCTGACTGTAAATGCTCCTGATACTC
TCTGCGAACACCTCCAGATAAATGATGGGGAGCTGATTGTTGTCCAGCTAACGCCTG
GATACTGTGCTCCTGAAGGATCTTACCACAGCACTGTGAACCAAGTACCACTGGGTT
GTAAGCCTTGCATATGTACACAGTTTGAGCTGGAGATCCTGTCCATGCAGAACGTGA
ATGGAGAGCTACAGAATGGGAACTGTTGTGGTGGAGTCCGGAACCCTGGCGACCGC
AAGTGCACCCGCGACGAGTGTGATACGTACTTCAAAGTGTGCCTCAAGGAGTATCA
GTCCCGCGTCACTGCCGGGGGACCCTGCAGCTTCGGCTCAGGGTCTACGCCTGTCAT
CGGGGGTAACACCTTCAATCTCAAGGCCAGCCGTGGCAACGACCGTAATCGCATCG
TACTGCCTTTCAGTTTCGCCTGGCCGAGGTCCTACACTTTGCTG
GTGGAGGCCTGGGATTCCAGTAATGACACTATTCAACCTGATAGCATAATTGAAAA
GGCTTCTCACTCAGGCATGATAAACCCTAGCCGGCAATGGCAGACACTGAAACAAA
ACACAGGGATTGCCCACTTCGAGTATCAGATCCGAGTGACCTGTGATGACCACTACT
ATGGCTTTGGCTGCAATAAGTTCTGTCGTCCCAGAGATGACTTCTTTGGACATTATGC
CTGTGACCAGAACGGCAACAAAACTTGCATGGAAGGCTGGATGGGTCCTGATTGCA
ACAAAGCTATCTGCCGACAGGGCTGCAGTCCCAAGCATGGGTCTTGTAAACTTCCAG
GTGACTGCAGGTGCCAGTACGGTTGGCAGGGCCTGTACTGCGACAAGTGCATCCCG
CACCCAGGATGTGTCCACGGCACC
TGCAATGAACCCTGGCAGTGCCTCTGTGAGACCAACTGGGGTGGACAGCTCTGTGAC
AAAGATCTGAATTACTGTGGGACTCATCAGCCCTGTCTCAACCGGGGAACATGTAGC
AACACTGGGCCTGACAAATACCAGTGCTCCTGCCCAGAGGGCTACTCGGGCCCCAA
CTGTGAAATTGCTGAGCATGCTTGTCTCTCTGACCCCTGCCATAACCGAGGCAGCTG
CAAGGAGACCTCCTCAGGCTTTGAGTGTGAGTGTTCTCCAGGCTGGACTGGCCCCAC
GTGTTCCACAAACATCGATGACTGTTCTCCAAATAACTGTTCCCATGGGGGCACCTG
CCAGGATCTGGTGAATGGATTCAAGTGTGTGTGCCCGCCCCAGTGGACTGGCAAGA
CTTGTCAGTTAGATGCAAATGAGTGCGAGGCCAAACCTTGTGTAAATGCCAGATCCT
GTAAGAATCTGATTGCCAGCTACTACTGTGATTGCCTTCCTGGCTGGATGGGTCAGA
ACTGTGACATAAATATCAATGACTGCCTTGGCCAGTGTCAGAATGACGCCTCCTGTC
GGGATTTGGTTAATGGTTATCGCTGTATCTGTCCACCTGGCTATGCAGGCGATCACT
GTGAGAGAGACATCGATGAGTGTGCTAGCAACCCCTGCTTGAATGGGGGTCACTGT
CAGAATGAAATCAACAGATTCCAGTGTCTCTGTCCCACTGGTTTCTCTGGAAACCTC
TGTCAGCTGGACATCGATTACTGCGAGCCCAACCCTTGCCAGAATGGCGCCCAGTGC
TACAATCGTGCCAGTGACTATTTCTGCAAGTGCCCCGAGGACTAT
GAGGGCAAGAACTGCTCACACCTGAAAGACCACTGCCGTACCACCACCTGCGAAGT
GATTGACAGCTGCACTGTGGCCATGGCCTCCAACGACACGCCTGAAGGGGTGCGGT
ATATCTCTTCTAACGTCTGTGGTCCCCATGGGAAGTGCAAGAGCCAGTCGGGAGGCA
AATTCACCTGTGACTGTAACAAAGGCTTCACCGGCACCTACTGCCATGAAAATATCA
ACGACTGCGAGAGCAACCCCTGTAAAAACGGTGGCACCTGCATCGATGGCGTTAAC
TCCTACAAGTGTATCTGTAGTGACGGCTGGGAGGGAGCGCACTGTGAGAACAACAT
AAATGACTGTAGCCAGAACCCTTGTCACTACGGGGGTACATGTCGAGACCTGGTCA
ATGACTTTTACTGTGACTGCAAAAATGGCTGGAAAGGAAAGACTTGCCATTCCCGTG
ACAGCCAGTGTGACGAAGCCACGTGTAATAATGGTGGTACCTGCTATGATGAAGTG
GACACGTTTAAGTGCATGTGTCCCGGTGGCTGGGAAGGAACAACCTGTAATATAGCT
AGAAACAGTAGCTGCCTGCCGAACCCCTGTCATAATGGAGGTACCTGCGTGGTCAAT
GGAGACTCCTTCACCTGTGTCTGCAAAGAAGGCTGGGAGGGGCCTATTTGTACTCAA
AATACCAACGACTGCAGTCCCCATCCTTGTTACAATAGCGGGACCTGTGTGGACGGA
GACAACTGGTATCGGTGCGAATGTGCCCCGGGTTTTGCTGGGCCAGACTGCAGGATA
AACATCAATGAGTGCCAGTCTTCCCCTTGTGCCTTTGGGGCCACCTGTGTGGATGAG
ATCAATGGCTACCAGTGTATCTGCCCTCCAGGACATAGTGGTGCCAAGTGCCATGAA
GTTTCAGGGCGATCTTGCATCACCATGGGGAGAGTGATACTTGATGGGGCCAAGTG
GGATGATGACTGTAACACCTGCCAGTGCCTGAATGGACGGGTGGCCTGCTCCAAGG
TCTGGTGTGGCCCGAGACCTTGCAGGCTCCACAAAAGCCACAATGAGTGCCCCAGT
GGGCAGAGCTGCATCCCGGTCCTGGATGACCAGTGTTTCGTGCGCCCCTGCACTGGT
GTTGGCGAGTGTCGGTCCTCCAGCCTCCAGCCAGTGAAGACCAAGTGCACATCTGAC
TCCTATTACCAGGATAACTGTGCAAACATCACTTTCACCTTTAACAAAGAGATGATG
TCTCCAGGTCTTACCACCGAACACATTTGCAGCGAATTGAGGAATTTGAATATCCTG
AAGAATGTTTCTGCTGAATATTCGATCTACATAGCCTGTGAGCCTTCCCTGTCAGCA
AACAATGAAATACACGTGGCCATCTCTGCAGAAGACATCCGGGATGATGGGAACCC
TGTCAAGGAAATTACCGATAAAATAATAGATCTCGTTAGTAAACGGGATGGAAACA
GCTCACTTATTGCTGCGGTTGCAGAAGTCAGAGTTCAGAGGCGTCCTCTGAAAAACA
GAACAGATGGGAATTCGATATCGGCCATGGTTAGATCTGGTTGTAAGCCTTGCATAT
GTACAGTCCCAGAAGTATCATCTGTCTTCATCTTCCCCCCAAAGCCCAAGGATGTGC
TCACCATTACTCTGACTCCTAAGGTCACGTGTGTTGTGGTAGACATCAGCAAGGATG
ATCCCGAGGTCCAGTTCAGCTGGTTTGTAGATGATGTGGAGGTGCACACAGCTCAGA
CGCAACCCCGGGAGGAGCAGTTCAACAGCACTTTCCGCTCAGTCAGTGAACTTCCCA
TCATGCACCAGGACTGGCTCAATGGCAAGGAGTTCAAATGCAGGGTCAACAGTGCA
GCTTTCCCTGCCCCCATCGAGAAAACCATCTCCAAAACCAAAGGCAGACCGAAGGC
TCCACAGGTGTACACCATTCCACCTCCCAAGGAGCAGATGGCCAAGGATAAAGTCA
GTCTGACCTGCATGATAACAGACTTCTTCCCTGAAGACATTACTGTGGAGTGGCAGT
GGAATGGGCAGCCAGCGGAGAACTACAAGAACACTCAGCCCATCATGGACACAGAT
GGCTCTTACTTCGTCTACAGCAAGCTCAATGTGCAGAAGAGCAACTGGGAGGCAGG
AAATACTTTCACCTGCTCTGTGTTACATGAGGGCCTGCACAACCACCATACTGAGAA
GAGCCTCTCCCACTCTCCTGGTAAATGA
IL-2 signal sequence-(1-60 bases)
OX40L-extracellular domain-(61-510 bases)
Fc-Linker-(511-534 bases)
Jagged1 extracellular domain-(535-3636 bases)
mouse IgG1-Fc2 sequences-(3667-4335 bases)
Mouse OX40L-JAG1-Fc Chimera protein sequence
(SEQ ID NO: 11)
QLSSSPAKDPPIQRLRGAVTRCEDGQLFISSYKNEYQTMEVQNNSVVIKCDGLYIIYLKG
SFFQEVKIDLHFREDHNPISIPMLNDGRRIVFTVVASLAFKDKVYLTVNAPDTLCEHLQIN
DGELIVVQLTPGYCAPEGSYHSTVNQVPL
GCKPCICTQFELEILSMQNVNGELQNGNCCGGVRNPGDRKCTRDECDTYFKVCLKEYQ
SRVTAGGPCSFGSGSTPVIGGNTFNLKASRGNDRNRIVLPFSFAWPRSYTLLVEAWDSSN
DTIQPDSIIEKASHSGMINPSRQWQTLKQNTGIAHFEYQIRVTCDDHYYGFGCNKFCRPR
DDFFGHYACDQNGNKTCMEGWMGPDCNKAICRQGCSPKHGSCKLPGDCRCQYGWQG
LYCDKCIPHPGCVHGTCNEPWQCLCETNWGGQLCDKDLNYCGTHQPCLNRGTCSNTG
PDKYQCSCPEGYSGPNCEIAEHACLSDPCHNRGSCKETSSGFECECSPGWTGPTCSTNID
DCSPNNCSHGGTCQDLVNGFKCVCPPQWTGKTCQLDANECEAKPCVNARSCKNLIASY
YCDCLPGWMGQNCDININDCLGQCQNDASCRDLVNGYRCICPPGYAGDHCERDIDECA
SNPCLNGGHCQNEINRFQCLCPTGFSGNLCQLDIDYCEPNPCQNGAQCYNRASDYFCKC
PEDYEGKNCSHLKDHCRTTTCEVIDSCTVAMASNDTPEGVRYISSNVCGPHGKCKSQSG
GKFTCDCNKGFTGTYCHENINDCESNPCKNGGTCIDGVNSYKCICSDGWEGAHCENNIN
DCSQNPCHYGGTCRDLVNDFYCDCKNGWKGKTCHSRDSQCDEATCNNGGTCYDEVD
TFKCMCPGGWEGTTCNIARNSSCLPNPCHNGGTCVVNGDSFTCVCKEGWEGPICTQNT
NDCSPHPCYNSGTCVDGDNWYRCECAPGFAGPDCRININECQSSPCAFGATCVDEINGY
QCICPPGHSGAKCHEVSGRSCITMGRVILDGAKWDDDCNTCQCLNGRVACSKVWCGPR
PCRLHKSHNECPSGQSCIPVLDDQCFVRPCTGVGECRSSSLQPVKTKCTSDSYYQDNCA
NITFTFNKEMMSPGLTTEHICSELRNLNILKNVSAEYSIYIACEPSLSANNEIHVAISAEDIR
DDGNPVKEITDKIIDLVSKRDGNSSLIAAVAEVRVQRRPLKNRTDGNSISAMVRSGCKPC
ICTVPEVSSVFIFPPKPKDVLTITLTPKVTCVVVDISKDDPEVQFSWFVDDVEVHTAQTQP
REEQFNSTFRSVSELPIMHQDWLNGKEFKCRVNSAAFPAPIEKTISKTKGRPKAPQVYTIP
PPKEQMAKDKVSLTCMITDFFPEDITVEWQWNGQPAENYKNTQPIMDTDGSYFVYSKL
NVQKSNWEAGNTFTCSVLHEGLHNHHTEKSLSHSPGK
OX40L extracellular domain-(1-150 amino acids)
Fc linker-(151-158 amino acids)
Jagged1 extracellular domain-(159-1192 amino acids)
mouse IgG1-Fc2 sequences-(1203-1424 amino acids)

1. A chimeric polypeptide comprising a first and a second polypeptide, wherein one of the polypeptides is an OX40L polypeptide and one of the polypeptides is a Jagged-1 polypeptide.

2. The chimeric polypeptide of claim 1 further comprising a linker.

3. The chimeric polypeptide of claim 1 wherein the first polypeptide is an OX40L polypeptide and the second polypeptide is a Jagged-1 polypeptide.

4. The chimeric polypeptide of claim 1 wherein the first polypeptide is a Jagged-1 polypeptide and the second polypeptide is an OX40L polypeptide.

5. The chimeric polypeptide of claim 1 wherein the OX40L polypeptide comprises the extracellular domain of OX40L or fragment thereof and the Jagged-1 polypeptide comprises the extracellular domain of Jagged-1 or fragment thereof.

6. The chimeric polypeptide of claim 1 wherein the protein further comprises a Fc region of an immunoglobulin.

7. The chimeric polypeptide of claim 6 wherein the Fc domain comprises the CH2 and CH3 regions of the IgG heavy chain and the hinge region.

8. The chimeric polypeptide of claim 1 wherein the linker comprises 10 amino acids from human immunoglobulin G1 hinge region.

9. The chimeric polypeptide of claim 1 wherein the linker comprises a polypeptide having SEQ ID NO: 13 or SEQ ID NO: 42.

10. A method of expanding T-regulatory cells comprising co-culturing said T-regulatory cells with the chimeric polypeptide of claim 1.

11. A method of treating an autoimmune disease in a patient in need of such treatment comprising administering to the patient a therapeutically effective amount of the chimeric polypeptide of claim 1.

12. The method of claim 11 wherein the autoimmune disease is an autoimmune thyroid disease.

13. The method of claim 11 wherein the autoimmune thyroid disease is Grave's disease or Hashimoto disease.

14. The method of claim 11 wherein the autoimmune disease is Type 1 Diabetes mellitus.

15. The method of claim 11 wherein said patient is a human patient.
